(19) United States
(12) Patent Application Publication
(10) Pub. No.: US 2014/0046606 A1
(43) Pub. Date: Feb. 13, 2014

(54) METHODS AND APPARATUS FOR MULTIBEAM DOPPLER ULTRASOUND DISPLAY

(71) Applicant: Stephen Robin GARDINER, London Greater London (GB)

(72) Inventors: Hans Torp, Trondheim (NO); Torbjorn Hergum, Trondheim (NO)

(73) Assignee: NORWEIGIAN UNIVERSITY OF SCIENCE AND TECHNOLOGY (NTNU), Tronheim (NO)

(21) Appl. No.: 14/007,479

(22) PCT Filed: Mar. 23, 2012
(86) PCT No.: PCT/GB12/50654

§ 371 Date: Oct. 23, 2013

(30) Foreign Application Priority Data
Mar. 25, 2011 (GB) 1105074.7
Mar. 25, 2011 (GB) 1105075.4

(51) Int. Cl. A61B 8/06

(52) U.S. Cl. CPC A61B 8/06
USPC 702/54

(57) ABSTRACT

There is provided a method for monitoring fluid flow, the method including: acquiring a pulse-echo signal from each of a plurality of spatially adjacent regions; for each of the pulse echo signals, forming a velocity spectrum; combining the plurality of velocity spectra into a composite data set which contains both spatial data and velocity data; and displaying the composite data set so as to present at least one aspect of the velocity data with at least one aspect of the spatial data. By combining both spatial and spectral velocity data into a composite data set and displaying aspects of the spatial data with aspects of the velocity data, the clinician can easily gauge in a quantitative or semi-quantitative way, both the size and velocities of fluid flows. In particular, the technique is useful for assessing valvular regurgitation jets in cardiac blood flows.

The invention relates to apparatus and methods for assessing fluid flow through an orifice using spectral pulsed wave Doppler ultrasound. In specific, preferred embodiments, the invention relates to the measurement and grading of blood flow through orifices in a human or animal body. More specifically, the invention may be used to measure and grade regurgitant blood flow jets in the valves of the heart and may be used to measure and grade stenotic valves or stenotic veins and/or arteries.

In some circumstances, blood flow through the vessels and organs of the body can be restricted or altered. For example, veins and arteries can become narrowed or blocked. The same can happen with valves in the heart. Also, holes in the valves can prevent correct operation of the valve by allowing backflow of fluid when the valve is in the closed position. Such anomalies may be congenital or acquired. These situations can be problematic. For example a regurgitant jet in one of the valves in the heart reduces the efficiency of the heart. As blood leaks back through the valve, the heart has to pump harder and/or faster to achieve a given level of circulation. This puts extra strain on the heart that can eventually lead to heart failure.

Diagnosing the above problems presents difficulties to the clinician. The organs in question are not readily accessible for examination and are also vital for life. Therefore intervention should ideally be minimised. It is also highly desirable for the clinician to be able to identify the extent of any abnormalities as a decision has to be made as to whether corrective intervention is necessary. For example heart surgery is a serious procedure which is only undertaken where the seriousness of the situation merits it. The clinician not only has to be able to identify the existence of an abnormality but also has to be able to grade it or evaluate its severity.

Ultrasound is a useful tool for assessing internal organs non-invasively. Several ultrasound techniques are available for imaging the internal structure of the body as well as detecting and assessing fluid flows within the body.

The most robust way to identify a hole or a leak in a valve is to detect a flow of fluid therethrough. Large holes can be detected with regular B-mode ultrasound imaging, but smaller leakages cannot be robustly detected by B-mode imaging due to its limited resolution, but they can still be found using Doppler techniques to identify fluid flow.

Doppler techniques in ultrasound are known and have been available for many years. The main techniques currently in use are Continuous Wave (CW) Doppler, Pulsed Wave (PW) Doppler and Colour Flow (CF) Doppler.

Continuous Wave Doppler simultaneously transmits an ultrasound signal and receives a backscattered (reflected) signal and monitors the frequency shift in the received signal so as to identify the velocities of moving scatterers in the path of the beam. As the transmitted signal is continuous, the receiver cannot identify the spatial location of the scatterers (i.e. their depth along the beam). However CW Doppler does not suffer from aliasing and can be used to detect a full range of typical and atypical blood flow velocities along the beam. It is particularly useful for identifying peak velocities accurately.

Pulsed Wave Doppler transmits short pulses of ultrasound, each pulse comprising a few wavelengths of oscillation. A particular depth (or range) along the beam direction can be selected by selecting a particular time interval in which to examine the echo signal after a transmit pulse. By increasing the time delay after the transmit pulse, the echo signal from regions further from the transducer can be examined. In normal operation the echoes from one pulse are observed before transmission of the next pulse. Therefore the depth of the region being examined affects the Pulse Repetition Frequency (PRF), with deeper regions having a greater pulse-echo round trip time and therefore requiring lower PRFs. Limitations on the PRF mean that even moderate flow rates can exceed the Nyquist sampling limit which leads to aliasing. For example, normal blood flow does not typically exceed 1 metre per second, but a mitral regurgitation jet can easily exceed 5 metres per second which would lead to aliasing at normal PRFs.

PW Doppler can also be performed in a High PRF (HPRF) mode in which new pulses are sent out without first waiting for the returning echo signals from previous pulses. This reduces aliasing by increasing the detectable velocity range, but at the expense of introducing spatial ambiguity along the beam length.

The signal processor typically combines and processes the signals from around 60-100 pulses in order to produce a power-velocity spectrum. The output of PW Doppler is typically displayed on a monitor with time along one axis (horizontal) and velocity along the other axis (vertical). Each pixel corresponds to a velocity bin (i.e. a Doppler frequency bin) and the brightness of the pixel represents the signal power in that velocity bin at that time. This provides a very detailed analysis of the flow within the region under examination and is a very useful tool for the clinician.

Colour Flow Doppler imaging uses similar techniques to PW Doppler, but provides a less detailed velocity analysis of any given region. The aim of CF Doppler imaging is to give an overall visual output which shows spatial and temporal variations in flow. Therefore instead of providing a detailed velocity spectrum analysis of one small location it provides an average velocity for a plurality of locations covering a larger spatial area. The output is displayed with colour representing different velocities, typically with red indicating flow towards the transducer and blue indicating flow away from the transducer. The colour data is typically output on top of a standard B-mode image so that the flows can readily be identified and interpreted in relation to the surrounding tissues.

A modern ultrasound apparatus can typically display several of the above measurement techniques simultaneously, but the techniques are not performed simultaneously. Rather they are interleaved in a time sharing manner. For example a B-mode image can be interleaved with a CF image by allocating alternate time slices to B-mode and CF imaging. This gives the impression of simultaneous performance, but in reality both techniques are compromised in that neither of them has full use of the transducer and therefore the frame rates are significantly reduced. The time sharing arrangement is flexible. For example if the sonographer has requested Colour Flow and B-mode imaging, it is most likely that the CF technique is of more interest, so more transducer time and more processing time can be allocated to it. For example, one B-mode frame could be provided for every two CF frames. Different interleaving ratios may of course be used. For example, the B-mode frame rate can be lowered significantly in situations where little tissue movement is expected (e.g. within an artery/vein), allowing a corresponding improvement in CF frame rate. Spectral PW Doppler acquisition can similarly be interleaved with similar reductions in the quality of data obtained. The gaps in the PW Doppler spectrum caused by interleaving are typically filled using a missing signal estimator.

As mentioned above, the goal of the clinician is to quickly identify and assess the severity of any abnormal fluid flows. Spectral PW Doppler and Colour Flow Doppler are useful in identifying the presence of abnormal flows. These may typically show up as high velocity flows in abnormal directions. For example a mitral regurgitation jet will show up on a colour flow image as a jet of fluid passing backwards through the mitral valve when the left ventricle contracts to drive fluid out past the aortic valve and round the body. On a PW spectrum taken at a point within the vena contracta of the jet (just behind the valve), it will show up as a period of backward flow with a velocity spectrum indicative of a fairly laminar flow.

Whilst these techniques are good for identification of the abnormal flow, they do not provide reliable information as to the severity of the problem, e.g. the size of the hole or the volume of blood flowing through it. The Colour Flow image does not provide accurate or reliable information regarding the velocities in the jet and the apparent size of the jet can vary with the gain setting of the equipment. The spectral PW Doppler gives more detailed and more accurate information on the velocities within the jet, but provides little information on the size of the jet. Neither of these techniques therefore provide a reliable indicator as to the severity of the problem.

A number of techniques have been developed which attempt to quantify the severity of such problems. One such technique is the Proximal Isovelocity Surface Area (PISA) technique, but this relies on a number of geometrical assumptions which make it inaccurate, especially for eccentric jets or where multiple jets exist. Another technique used in assessment of valvular regurgitation is to measure inflow and outflow either side of the valve and thus calculate the volume of backflow through the valve. For example, to assess a mitral regurgitation, the outflow through the aorta can be subtracted from the inflow through the open mitral valve. However this relies on a number of error prone measurements and is therefore not an accurate indicator.

U.S. Pat. No. 6,544,181 describes a technique for estimating the area of an orifice and also calculating the volume of flow therethrough. The technique involves using a PW ultrasound measurement beam steered to within the laminar flow of the vena contracta of the orifice and obtaining a power-velocity spectrum in that region. The power velocity spectrum is integrated to give power which is proportional to the instantaneous cross section of the orifice. An absolute value for area can be obtained by also applying a reference beam of known cross-sectional area within the area of the measurement beam and performing a similar analysis so as to calibrate the measurement reading. Velocity information can also be combined with the calculated area to provide a volume flow rate through the orifice. To ensure that only laminar flow is taken into consideration, the velocity spectrum is filtered either side of the velocity at peak power, i.e. the lower power velocities are excluded from the power-integral calculation.

In US 2009/0043208, another technique for estimating the cross-sectional area of an orifice is provided. In this technique, an array of multiple small ultrasound beams is directed at the orifice, the beams being spatially adjacent to one another. Each beam is smaller than the size of the orifice. Together, the array can cover the whole area of the orifice. One reference beam is selected from the array, e.g. the beam with the highest power. The cross-sectional area of the orifice can then be calculated based on the power of the reference beam, the power of the composite beam (formed from all beams in the array) and the beam profiles of the reference beam and the composite beam. This technique has the advantage that it gives a more spatially homogeneous measurement of the Doppler power from the leakage. Also it does not use spectral analysis to isolate the signal from the laminar jet, as it uses a clutter filter and Doppler power estimator as is commonly used for color flow imaging. An additional benefit of this technique is that the array of beams can be used to provide a measure of the geometry of the orifice.

The above two techniques can be used to provide a quantitative assessment of an orifice. However the quantitative output, although useful, is not always easy for the sonographer to monitor and digest during a sonographic examination. For example, the orifice area is instantaneous and can be re-calculated continuously in time. The orifice area may in fact vary in time, for example with the opening and closing of a valve. The sonographer therefore has to select one or more time points at which to assess the orifice area. A sample of data can be recorded and the sonographer can look back through the data to select the relevant points, but it would be advantageous to have a semi-quantitative or qualitative indication of the severity of the anomalous flow in real time which could be easily visualised. This would allow the sonographer to identify and grade problems more rapidly and would facilitate adjusting the ultrasound probe in relation to the orifice in real time so as to obtain the best data possible. This can be particularly difficult in the case of the heart where the patient may be moving (e.g. breathing) and the organ itself is moving (beating).

According to the invention there is provided a method for monitoring fluid flow, the method comprising: acquiring a pulse-echo signal from each of a plurality of spatially adjacent regions; for each of the pulse-echo signals, forming a velocity spectrum; combining the plurality of velocity spectra into a composite data set which contains both spatial data and velocity data; and displaying the composite data set so as to present at least one aspect of the velocity data with at least one aspect of the spatial data.

Pulse-echo signals may be acquired by transmitting pulsed sound energy (typically ultrasound) from a transmitter into the region of interest and acquiring the echoes by a receiver. Echoes are received when the sound energy is backscattered by scatterers (e.g. red blood cells) in the path of the transmitted pulse.

The transmitter and receiver may be the same or different devices. In preferred embodiments they are one or more ultrasound transducers. The pulses transmitted into the region of interest may be a stream of regularly spaced pulses. Each transmitted pulse typically comprises a plurality of wavelengths of oscillation transmitted at a transducer transmission frequency. The pulses are transmitted at regular intervals determined by the pulse repetition frequency (PRF). As the pulses are finite in length, each pulse has a bandwidth around the transducer transmission frequency.

In this specification, the term “pulse-echo signal” includes a series of echoes received from a series of many pulses.

As the received pulse-echo signal is acquired from several spatially adjacent regions, the acquired data set contains spatial information about the region of interest. Further, as the acquired data is used to form a velocity spectrum for each region, the acquired data set contains detailed velocity information. By combining the pulse-echo signals from several spatially adjacent regions into a single data set, the output can be displayed so as to simultaneously display aspects of both the spatial information and the velocity information derived from the received signals. This allows both aspects of the data to be viewed, analysed and evaluated rapidly and efficiently.

The pulse-echo signal acquired for each region is used to form a full Doppler power-velocity spectrum which can detect the full spread of velocities within the target region (subject to aliasing limitations). Forming a full power-velocity spectrum for each spatial region allows detailed analysis to be performed on the combined data set.

A power velocity spectrum represents the signal power received as a function of velocity and is typically obtained by means of signal processing of a received time domain signal. The signal processing is typically performed digitally on a sampled receive signal and the velocity spectrum is typically a discrete spectrum with the data divided into a plurality of discrete velocity bins.

The ultrasound unit may be a linear array, but is preferably a two-dimensional array (of the type used for 3D ultrasound), i.e. comprising a two-dimensional array of transducer elements for transmitting pulses and receiving echoes. Similarly, the spatially adjacent regions may be a linear array of regions (i.e. a linear array of beams), but is preferably a two dimensional array of regions (i.e. a two dimensional array of beams). The acquisition of data from the plurality of regions/beams may be done sequentially (i.e. separate transmit pulses are used for each region) or it may be done with parallel beam forming (i.e. a single transmit pulse is used to insonify a region of interest, and a plurality of receive beams are formed in parallel from the received pulse-echo signals using standard beam forming techniques such as delay and sum techniques). Parallel beamforming allows simultaneous acquisition of pulse-echo signals from multiple spatial regions. This can for instance be used to increase the frame rate. The two techniques (sequential and parallel beam forming) may be combined to cover the full array of spatial regions. For example, the full set of regions may be divided into blocks of adjacent regions each of which can be interrogated with parallel receive beams. The blocks can then be interrogated sequentially so as to form a complete data set for all spatial regions.

In preferred embodiments, the step of combining the plurality of velocity spectra into a composite data set comprises combining the spectra into a composite velocity spectrum.

Velocity spectra are well known to sonographers and clinicians who are familiar with viewing and interpreting them. A velocity spectrum is obtained by using the Doppler principle whereby the frequency of a backscattered wave is altered from the transmitted frequency according to the velocity of the scatterer. By analysing the received frequencies compared with the transmitted frequencies, a spectrum of scatterer velocities can be obtained. During the analysis, the velocities may be collected together into velocity bins (Doppler frequency bins), i.e. each bin corresponding to a small velocity range. Each velocity bin stores the amount of received signal power corresponding to energy from scatterers travelling within the velocity range of that bin. Accordingly, the signal power within a velocity bin is related to the number of scatterers travelling at velocities within the velocity range of that bin.

By combining the composite data set into the form of a composite velocity spectrum, the data is combined into a form which can be displayed to the end users (e.g. sonographers and clinicians) so that it is easily recognizable and can readily be interpreted. As the general format of the displayed data is familiar to the users, it will be easier for them to learn how to interpret the new data while retaining the familiar techniques and assessments.

In some preferred embodiments, the composite velocity spectrum comprises a plurality of velocity bins each corresponding to a range of velocities, and the step of displaying the composite velocity spectrum includes displaying a representation of each velocity bin in the composite velocity spectrum with a colour that depends on the number of pulse-echo signals intersecting with the fluid flow.

In this manner, the velocity aspects of the composite data set are displayed in the form of a normal velocity spectrum while the spatial aspects of the composite data set are displayed using colour added to (superimposed on) the velocity spectrum. Using colour to denote the number of pulse-echo signals intersecting the fluid flow (i.e. the number of receive beams intersecting the fluid flow rather than intersecting the surrounding vessel walls) gives a non-spatial representation of the spatial information, i.e. the spatial information from the plurality of spatially adjacent regions can be represented by a single value (or colour). The number of pulse-echo signals intersecting with the fluid flow is related to the cross sectional area of the fluid flow.

As described above, a normal velocity spectrum is displayed by plotting representations of the velocity bins along an axis (plotting typically involves illuminating pixels on a display, but other forms of plotting may also be used). Typically the data is displayed with the brightness of the plotted representation being determined according to the power received in that velocity bin. Thus brighter regions of the display indicate velocities which are strongly present in the received signal data whereas darker regions of the display indicate velocities which are only weakly present or are absent altogether from the received signal data.

Usually the velocity spectrum is plotted as a function of time, i.e. a velocity spectrum is plotted at each of a plurality of time points along a time axis. Each velocity spectrum represents data acquired over a certain time period. That time period may be a discrete and separate time period during which the received signal is sampled for each time point, but more usually the time period is based on a sliding time-window whereby when new sampled data is acquired, the oldest sampled data in the set is dropped. Typically the data used for each velocity spectrum (i.e. for each time point) are based on the received signal data for around 60-100 transmitted pulses. This amount of data is necessary to provide a velocity spectrum with adequate velocity resolution. By contrast, for example, Colour Flow Doppler imaging provides only an average velocity based on data from around 8-12 transmitted pulses.

Displaying the composite data set in this way means that the velocity information is easily recognisable in its usual format while the spatial information has been amalgamated into a single representation which is superimposed on the velocity information.

Preferably the composite velocity spectrum comprises a plurality of velocity bins each corresponding to a range of velocities and, in association with each velocity bin, the spectrum contains a representation of the number of individual region spectra in which the received signal power in that velocity bin is above a threshold value (i.e. the number of spatial regions in which the power-velocity spectrum for that region has a received signal power in that velocity bin above the threshold value).

In such embodiments, the composite data set indicates for each velocity bin the number of receive beams which have power falling above the threshold value. Thus the data indicate the number of receive beams (i.e. the spatial data) which are showing significant fluid flow in that velocity range. Thus the display of the data can provide a simple indication of the size of a particular fluid flow. In the case of regurgitant jets in heart valves, the jet will typically be identified by a region of high velocity flow. Thus, the velocity data of the composite data set will show significant received signal power in the velocity bins for higher velocities. The threshold is preferably set so as to isolate the particular velocity bins of interest, in this case the higher velocities. By counting and displaying the number of receive beams with power above the threshold, the spatial data from the composite data set can be incorporated into the display and can give an immediate indication of the size of the jet. For example, a larger jet will cover a larger number of spatial regions and thereby will indicate higher velocities in a larger number of receive beams (and thus more spectra with above-threshold power in the higher velocity bins). In the case of leaking heart valves, representing the data in this way provides an immediate and real-time visual indication of the severity of the leakage.

The threshold value may be the same for all velocity bins. However preferably the threshold value is different for different velocity bins. In particular, as the velocities increase, the transit-time effect of the transmitted pulses reduces the signal energy density. Therefore the same threshold will not necessarily be appropriate for all velocities and should be varied to take this effect into account.

The threshold will vary from situation to situation and will depend, amongst other things, on patient physiology as well as the nature of the problem being evaluated. The skilled person will be able to establish and set an appropriate threshold for any given circumstances. The setting of the threshold may be partially or fully automated in some embodiments. In others, the threshold may be controllable or settable by the sonographer, e.g. during the sonographic examination.

Preferably the method further comprises: displaying the composite velocity spectrum by plotting the velocity bins on a display such that a display characteristic of each velocity bin is determined from the number of individual region spectra in which the received signal power in that velocity bin is above a threshold value.

As mentioned above, displaying the composite data set in the form of a velocity spectrum with added spatial data provides the end user with a recognizable display and a familiar output which is easier to understand and analyse.

Preferably the display characteristic is the colour of the plotted velocity bin. This provides a visual indication which is easily added to the velocity spectrum without detracting from the familiar appearance of the velocity spectrum. As mentioned above, a typical velocity spectrum uses brightness to indicate received signal power, but is otherwise displayed using a single shade of colour, e.g. a single hue. Accordingly, the display characteristic used to represent the spatial data is preferably the colour of the plotted velocity bin.

Colours can be defined in a number of ways. In one scheme, a colour is defined by three variables: Hue, Saturation and Brightness. Any or all three of these may be varied in order to define the colours for displaying the spatial data. However, where power data is to be displayed (which will normally be the case), Brightness is preferably still used for that data (so as to retain the familiarity of the displayed output). Therefore Hue and Saturation can be varied for displaying spatial data. It will be appreciated that in other embodiments, Brightness may also be used, alone or in combination with Hue and/or Saturation as the display characteristic for representing the spatial data. In other embodiments other colour definitions may be used, e.g. RGB definitions instead of HSB definitions.

The colours used to represent different numbers may be arbitrarily selected. They may be discrete, unrelated colours, or they may be a scale of shades, e.g. a range of shades of red running from pale to intense. The scale may be colours with a single Hue, but with varying Saturation or it may be a scale of varying Hue (optionally with constant Saturation).

Preferably a visual scale is provided for the user to identify the particular colours or shades with the numerical data.

In some preferred embodiments, the composite velocity spectrum comprises a plurality of velocity bins each corresponding to a range of velocities, the plurality of regions are grouped into at least two groups, and in association with each velocity bin, the composite velocity spectrum comprises: a representation for each group of the total power in that velocity bin from all regions within that group. In other words, for each velocity bin of the composite velocity spectrum, there are a number of power representations (one for each group). The power representation for a group is obtained from the power (i.e. the power in the particular velocity bin being evaluated) in each of the individual spatial region spectra within that group.

Such embodiments provide an alternative way of representing the spatial data alongside the velocity data within the composite data set. The spatial regions are used to form two or more groups so that the power distribution in the velocity spectra (i.e. the velocity distributions) of one group can be compared with the power distribution in other group(s).

The representation for each group may be weighted according to the inverse of the number of regions within that group. For example, if there are N regions within a particular group, the total power obtained from those N regions may be weighted by a factor of 1/N. This weighting makes the signals from each group more directly comparable.

The regions may be divided into groups in any of a number of ways. In some embodiments all of the regions are assigned to a group (i.e. there are no regions which do not form part of a group). In other embodiments the subsets of regions which form the groups may be less than the whole set of regions. The groups may be of equal or unequal size. The members of a group may be spatially adjacent or spatially separate (i.e. each group need not be spatially contiguous, although in preferred embodiments they are). Also, the groups may overlap (i.e. a region may be a member of more than one group), although in preferred embodiments there is no overlap.

Preferably the method further comprises: displaying the composite velocity spectrum by plotting the velocity bins on a display such that a display characteristic of each velocity bin is determined from the total powers of each of the groups. In other words, within each velocity bin, the total power received from one group is combined with the total powers from the other group(s) to form the display characteristic.

Preferably the display characteristic is the colour of the plotted velocity bin. As above, the colour of the plotted velocity spectrum can be readily altered without affecting the familiar output format of the data, thus making the data more readily understandable and allowing for faster interpretation of the results as well as making it easier for users to understand and become familiar with the new data.

In some preferred embodiments, each group of regions is associated with a colour and the colour of the plotted velocity bin of the composite velocity spectrum is determined by combining the colours of the plurality of groups in amounts based on the group total power for that velocity bin. In other words, to form the colour for plotting a particular velocity bin, the power data is taken for that velocity bin from each group and these powers are combined so as to determine a colour. This may be done by adding together appropriate amounts of different colour vectors.

In certain preferred embodiments there are two groups of spatial regions and the colours associated with the two groups are complementary colours. Complementary colours are such that when added together in equal amounts they result in a neutral colour (i.e. a shade of grey). Some examples of complementary colours are: red and cyan, blue and yellow, green and magenta. When the colour of the displayed velocity bin is determined in this way, it indicates the balance between the received signal powers for the two groups. For example if the powers are the same, then the two group colours will be combined in equal measure and the colour will be neutral. If one group dominates, the two group colours will be combined in unequal measures and the combined colour will be more towards the colour of the dominant group. The operator can therefore easily identify which group dominates simply by looking at the colour of the output. For example, if using red/cyan, a strongly red colour or a strongly cyan colour would indicate one very dominant group. A weakly red or weakly cyan colour would indicate a slightly dominant group.

As mentioned above, the groups may be of equal or unequal size. In the case of unequal sizes, weighting may be used to balance the contributions from the different groups. The regions may be grouped into one group of one region and one group of all the remaining regions. Such an arrangement provides a comparison between a reference spatial region (i.e. a reference receive beam) and the rest of the spatial regions (i.e. the combination of the rest of the receive beams). This particular arrangement makes it particularly easy to identify the size of a fluid flow from the displayed colour. For example, if the reference beam (i.e. the first group) is selected as the beam with the greatest power (which can be assumed to be within the fluid flow), then the power from the remaining beams (i.e. the second group) can be compared with the reference beam to indicate how many of those beams are also within the fluid flow. When the group powers are weighted by the inverse of the number of beams (i.e. the power from the second group is weighted by a factor of 1/(N−1)), and when the colours for the two groups are selected to be complementary, the resultant colour will vary on a scale from the colour of the first group to a neutral colour. If the contribution from the second group is small, the overall colour will be close to that of the first colour. On the other hand, if the contribution from the second group is equal to that of the first group, the overall colour will be neutral. Therefore a neutral colour indicates a fluid flow of large cross-section as it indicates lots of power outside of the reference beam, whereas a colour close to that of the reference beam indicates a fluid flow of small cross-section as it indicates that there is little power outside of the reference beam. Thus in the displayed output, the colour gives an immediate indication of size of the fluid flow (e.g. the size of a regurgitant jet) and thus the size of the orifice under evaluation.

In other preferred embodiments the step of displaying the composite data set comprises: for each region, plotting on a display at a spatial location corresponding to the spatial location of that region a representation of that region, wherein a display characteristic of the plotted region is determined from the velocity data associated with that region.

In such embodiments, the spatial data provides the dominant presentation characteristic, i.e. the spatial data is illustrated with velocity data added to it rather than the other way round as described above. In such embodiments, the presentation of spatial data can be used to indicate the geometry of the observed fluid flow. For example, by identifying and displaying received signal data above a certain threshold velocity, the cross-sectional area of a fluid flow can be seen by distinguishing it from slower moving vessel walls and also from slow flow in ambiguous sample volumes in the case of HPRF acquisition. In the case of fluid flow through an orifice, such a display shows the geometry (i.e. the shape) of the orifice.

In some preferred embodiments the display characteristic is determined from the associated velocity data. In some preferred embodiments the display characteristic is determined from the signal power corresponding to each velocity bin. For example, the display characteristic may be determined based on the maximum velocity detected, the average velocity detected or the velocity at which maximum power is detected.

Preferably the display characteristic is the colour of the plotted region. The colour may be varied on a continuous scale representing a continuous scale of the appropriate velocity information or discrete colours may be associated with different portions of the velocity spectrum. For example the velocity spectrum could be divided into three groups (fast, medium and slow velocities) and the displayed colour for a particular region could be selected according to which of these groups receives the most power. With such a colouring scheme, for example red could indicate fast velocities, yellow medium velocities and green slow velocities. Thus the clinician can easily identify a large problematic jet from a large red area on the display.

It will be appreciated that the above schemes may be combined or displayed alongside each other. For example, in particularly preferred embodiments, the geometry view is displayed alongside either of the two velocity spectrum-based views. Alternatively all three views may be displayed simultaneously or may be selectable by the operator.

In preferred embodiments, the acquired signal data is filtered to remove low frequency components of the received signal. Such filters are often known an “clutter filters” as they remove low frequency signals reflected from slow moving fluid and other slow moving structures. For example, in the case of heart or other blood flow monitoring, low velocity blood and tissue structures (vessel walls, valves, etc.) generate strong unwanted signal which can be removed with a high pass clutter filter. The clutter filter is typically a digital filter, operating on sampled signal data, along the pulse (“slow time”) direction.

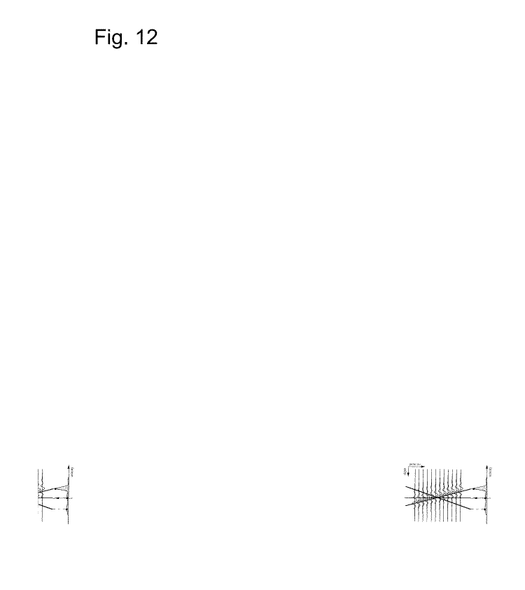

“Fast time” and “Slow time” are two time scales on which sampling takes place. “Fast time” is the time scale on which the receiver samples the incoming signal, i.e. for a single pulse, the echo would be sampled several times. “Slow time” is the time scale on which samples are taken for calculating a given velocity spectrum, i.e. one sample per pulse echo is selected from the “fast time” samples taken by the receiver to produce a “slow time” subset of samples. This gives a two-dimensional signal as is illustrated in FIG. 12. In FIG. 12 the slow time direction is labelled “Pulse no.”, and the corresponding “fast time” direction is labelled “depth”. The clutter filter is used along the slow-time dimension.

In preferred embodiments, the method may use the Velocity Matched Spectrum technique described in U.S. Pat. No. 5,662,115 for generating the velocity spectrum for each spatial region. This technique tracks the movement of the scatterer from pulse to pulse within the acquisition pulse stream by varying the sampling time of the receiver so as to match the change in position of the scatterer. By processing the signal data for a plurality of trial velocities, a velocity spectrum can be built up which has higher signal power where the trial velocity correlates with actual fluid velocities within the flow. This technique means that aliasing can be avoided without using High PRF (HPRF) techniques, or using HPRF with fewer ambiguous sample volumes. This will allow the use of a lower pulse repetition frequency than would otherwise be required to avoid aliasing, which will allow better signal to noise ratio in the estimates since the receiver will have more time to recover after pulse transmission and thus avoid saturation.

Thus, in preferred embodiments, the pulse-echo signals are acquired from a series of pulses, said pulses being transmitting at a series of pulse transmission times; and the method further comprises: for each of the pulse-echo signals, processing said signal to form a velocity power spectrum; wherein the step of processing comprises: for each of a plurality of velocities, sampling the pulse-echo signal at a series of sampling times, said sampling times being offset from said pulse transmission times by a variable offset which increases or decreases along the series according to the increasing or decreasing round-trip time from a potential scatterer moving at said velocity.

Although the Velocity Matched Spectrum technique does not itself suffer from velocity aliasing, the effect of the clutter filter does involve aliasing. The clutter filter removes power from the received signal, not just in a frequency band corresponding to the slow moving fluid and structures, but also in frequency bands around multiples of the Nyquist aliasing frequency. With conventional processing, this loss of signal power around the aliasing frequency leads to a complete absence of data in the velocity spectrum at those frequencies. By contrast, with the Velocity Matched Spectrum technique, some signal power for those velocities can still be obtained. Therefore, although the clutter filter causes signal loss, it does not completely obliterate the signal. This allows the shape of the spectrum to be seen (displayed) and assessed, thereby providing better data for the clinician to analyse.

Although the above-described techniques provide a good qualitative and semi-quantitative assessment of fluid flows, e.g. the severity of a valvular regurgitation, it is still further desirable to be able to quantify the fluid flow more precisely, e.g. to be able to calculate the volume flow rate of the fluid.

Preferably, therefore, the pulse-echo signals are acquired from a series of pulses, said pulses being transmitting at a series of pulse transmission times; and the method further comprises: filtering said pulse-echo signals to exclude low frequency signal information; and for each of the pulse-echo signals, processing said signal to form a velocity power spectrum; and adjusting said power spectrum to compensate for power lost in the filtering step, the adjustment being based on the characteristics of the filtering step; wherein the step of processing comprises: for each of a plurality of velocities, sampling the pulse-echo signal at a series of sampling times, said sampling times being offset from said pulse transmission times by a variable offset which increases or decreases along the series according to the increasing or decreasing round-trip time from a potential scatterer moving at said velocity.

Thus, by applying the velocity matched spectrum technique for spectrum formation, not all signal data is lost for velocities around the aliasing limit.

Further, as the signal loss is entirely due to the filter step, and the properties of the filter are known, the received signal can be accurately compensated to counteract the effect of the filter. Thus the original signal can essentially be reconstructed and thereby a quantitative measurement of the fluid flow can be obtained. Combining the accurate velocity information from the compensated velocity spectrum with the cross-sectional area of the fluid flow allows a volume flow rate of the fluid to be calculated.

Therefore the method may further comprise the step of: calculating a volume flow rate of the fluid flow based on the plurality of adjusted power spectra from the plurality of spatially adjacent regions. The calculation of the volume flow rate preferably involves the estimation or calculation of cross-sectional area of the fluid flow. Preferably an estimate of the cross-sectional area is acquired from the spatial information obtained by using a plurality of spatially adjacent regions.

In preferred embodiments, the step of adjusting the power spectrum includes using a signal model to model the system both with and without the filter and comparing the two models to determine signal power loss as a function of velocity due to the presence of the filter in the system. This technique will be discussed in more detail later, but essentially it isolates the effect of the filter, thereby allowing an accurate compensation for the effect that the filter has had on the received signal.

For some signal models, the signal model may take account of a plurality of system parameters, including the center frequency of the pulse, the pulse repetition frequency, pulse bandwidth and window function parameters. Preferably the step of filtering includes using a digital filter having a set of filter coefficients. Preferably the process is carried out repeatedly in real-time so as to output a real-time quantitative output characteristic of the fluid flow.

The signal model calculations may be carried out in advance of other measurements and the results stored, e.g. in a memory. For example the compensation function may be stored in a look-up table. Alternatively, as the signal model calculations are not very computationally intensive, the calculations may be performed on-the-fly at the time that they are required. Certain system parameters may change with user settings of the equipment, e.g. the PRF. Other parameters may be varied by the system itself as part of other processing functions. Likewise, the filter parameters may be varied (e.g. the cut-off frequency may be varied depending on the circumstances of the measurements being taken). Therefore the signal model calculations will need to be updated when the system parameters change.

In some preferred embodiments, the step of processing comprises: for each of a plurality of radial and lateral velocities, sampling more than one of said spatially adjacent pulse-echo signals at a series of sampling times, said sampling times being offset from said pulse transmission times by a variable offset which increases or decreases along the series according to the increasing or decreasing round-trip time from a potential scatter moving at said radial velocity, and wherein said samples are taken from the appropriate pulse-echo signal according to the expected lateral position of a potential scatterer moving at said lateral velocity. This enables tracking in the radial direction (towards or away from the transmitter) at the same time as tracking in the lateral directions (perpendicular to the radial direction).

Viewed form an alternative perspective, the invention provides a method for monitoring fluid flow through an orifice, the method comprising: for each of a plurality of spatially adjacent regions, insonifying the region with a pulsed ultrasound signal; receiving echoes of the pulsed ultrasound signal from the region; processing the received signal data to form a velocity spectrum for the region; combining the plurality of velocity spectra into a composite data set which contains both spatial data and velocity data; and displaying the composite data set so as to present at least one aspect of the velocity data with at least one aspect of the spatial data.

According to another aspect, the invention provides an apparatus for monitoring fluid flow, comprising: a transducer arranged to acquire a pulse-echo signal from each of a plurality of spatially adjacent regions; and a processor arranged to form a velocity spectrum for each of the pulse echo signals; combine the plurality of velocity spectra into a composite data set which contains both spatial data and velocity data; and display the composite data set so as to present at least one aspect of the velocity data with at least one aspect of the spatial data.

According to yet further aspects, the invention extends to use of the above apparatus and methods for monitoring fluid flow through valves within the circulatory system of a human or animal, in particular the valves of the heart (such as the mitral valve). In particularly preferred embodiments, the invention extends to use of the above apparatus and methods to monitor regurgitant jets in valves of the circulatory system of humans or animals. The invention likewise extends to monitoring of stenotic valves in the heart and to monitoring of stenotic veins and/or arteries.

According to yet a further aspect, the invention extends to a software product comprising instructions which when executed by a computer cause the computer to: acquire a pulse-echo signal from each of a plurality of spatially adjacent regions; for each of the pulse echo signals, form a velocity spectrum; combine the plurality of velocity spectra into a composite data set which contains both spatial data and velocity data; and display the composite data set so as to present at least one aspect of the velocity data with at least one aspect of the spatial data.

The software product may be in the form of a physical data carrier. The software product may comprise signals transmitted from a remote location.

According to yet a further aspect, the invention extends to a method of manufacturing a software product which is in the form of a physical data carrier, comprising storing on the data carrier instructions which when executed by a computer cause the computer to: acquire a pulse-echo signal from each of a plurality of spatially adjacent regions; for each of the pulse echo signals, form a velocity spectrum; combine the plurality of velocity spectra into a composite data set which contains both spatial data and velocity data; and display the composite data set so as to present at least one aspect of the velocity data with at least one aspect of the spatial data.

According to yet a further aspect, the invention extends to a method of providing a software product to a remote location by means of transmitting data to a computer at that remote location, the data comprising instructions which when executed by the computer cause the computer to: acquire a pulse-echo signal from each of a plurality of spatially adjacent regions; for each of the pulse echo signals, form a velocity spectrum; combine the plurality of velocity spectra into a composite data set which contains both spatial data and velocity data; and display the composite data set so as to present at least one aspect of the velocity data with at least one aspect of the spatial data.

It will be appreciated that all of the preferred features described above in relation to the method apply equally to the apparatus and to the uses of both the methods and apparatus as well as to the software.

The concept of quantifying a fluid flow using signals from a plurality of spatially adjacent regions and using the velocity matched spectrum technique is considered to be independently inventive.

Therefore according to a further aspect of the invention, there is provided a method of quantifying a fluid flow, the method comprising: transmitting a series of pulses, said pulses being transmitting at a series of pulse transmission times; acquiring a pulse-echo signal from each of a plurality of spatially adjacent regions; filtering said pulse-echo signals to exclude low frequency signal information; and for each of the pulse-echo signals, processing said signal to form a velocity power spectrum; and adjusting said power spectrum to compensate for power lost in the filtering step, the adjustment being based on the characteristics of the filtering step; wherein the step of processing comprises: for each of a plurality of velocities, sampling the pulse-echo signal at a series of sampling times, said sampling times being offset from said pulse transmission times by a variable offset which increases or decreases along the series according to the increasing or decreasing round-trip time from a potential scatterer moving at said velocity.

By varying the sampling times of the received pulse-echo signals so that they track a particular fluid velocity within the target region (i.e. using the Velocity Matched Spectrum technique), more velocity information can be extracted from the received signals and a velocity spectrum can be created which is less restricted by aliasing from the pulse repetition frequency (PRF). This technique means that aliasing can be avoided without using High PRF (HPRF) techniques, or using HPRF with fewer ambiguous sample volumes. This will avow the use of a lower pulse repetition frequency than would otherwise be required to avoid aliasing, which will allow better signal to noise ratio in the estimates since the receiver will have more time to recover after pulse transmission and thus avoid saturation.

An additional benefit of the Velocity Matched Spectrum technique is that, provided the pulse bandwidth is high enough, the information used in the power analysis of velocities around the conventional aliasing limit is spread over a larger frequency range, which means that the aliasing of the clutter filter does not exclude all the information for such velocities. As some of this information is retained, and the properties of the clutter filter are known, the magnitude of the missing information can be reconstructed, thereby allowing a quantitative analysis of the signal even around the conventional aliased filter band.

The use of a plurality of receive beams for a plurality of spatially adjacent regions means that a quantitative analysis can be provided for each separate receive beam. When the plurality of receive beams are directed at an orifice through which fluid flows, the plurality of beams can be used to analyse the cross-sectional area of the orifice. Combining this with the quantitatively accurate velocity spectra across that cross-sectional area, an accurate estimate can be obtained of fluid flow rate through the orifice. One particular benefit of this system and method is that the fluid flow rate through a regurgitative orifice in a heart valve, for example, is indicative of the severity of the pathology. Therefore providing an accurate measurement of fluid flow rate through the orifice provides an immediate indication of the severity of the orifice and is therefore a very useful tool for facilitating diagnoses of such problems.

Therefore, although the system can be used simply to provide a quantitatively accurate velocity power spectrum for each region, which may be useful in a variety of situations, some preferred embodiments are for measuring the cross sectional area and volume flow rate.

Preferably the method further comprises the step of: calculating a volume flow rate of the fluid flow based on the plurality of adjusted power spectra from the plurality of spatially adjacent regions.

Preferably the calculation of volume flow rate involves an estimate of a cross-sectional area through which the fluid flows. That area may simply be the area covered by the plurality of spatially adjacent regions or it may be a smaller area determined by a separate analysis of the power velocity spectra. For example, in the case of fluid flow through a tissue orifice, it can be determined whether each individual region falls within the orifice (generally experiencing high velocities) or outside the orifice (generally experiencing lower velocities as the tissue surrounding the orifice only moves slowly).

In preferred embodiments, the step of adjusting the power spectrum includes using a signal model to model the system both with and without the filter and comparing the two models to determine signal power loss as a function of velocity due to the presence of the filter in the system.

As will be described in more detail later, the signal model provides an expected response of the system to certain inputs (i.e. to certain flow velocities). By comparing the expected response of the system including the filter with the expected response of the system excluding the filter, the effect of the filter on the signal analysis can be determined. As the filter cuts out low frequency signal data, the filter reduces the power detected by the system at certain velocities whose Doppler frequencies correspond to the frequencies blocked by the filter or to corresponding aliased frequencies. As the filter is intended to block the frequencies in the non-aliased part of the signal, the received signal power for such velocities should be highly attenuated. However because of the use of the velocity matched spectrum technique to form the velocity power spectrum, the attenuation due to the filter is limited in the aliased portions of the spectrum and it can be compensated in accordance with the signal model.

In some preferred embodiments, the signal compensation is only performed on velocities above a certain threshold as it is not intended to compensate the signal for those velocities which were intended to be filtered out. In other words, the signal compensation is only required for velocities affected by the aliasing of the filter.

For some signal models, the signal model may take account of a plurality of system parameters, including the centre frequency of the pulse, the pulse repetition frequency, pulse bandwidth and window function parameters.

These parameters are used as inputs to the signal model and are used to calculate the expected response of the system to particular flow velocities.

Preferably the step of filtering includes using a digital filter having a set of filter coefficients.

Digital filters are particularly preferred as the signal processing is typically carried out digitally on a sampled signal. The filter coefficients define the frequency response of the filter and form a further input to the signal model which can then be used to determine the effect that the filter has on the expected output of the model.

Preferably the process is carried out repeatedly in real-time so as to output a real-time quantitative output characteristic of the fluid flow.

This is in line with the typical operation of a velocity power spectrum produced by a medical Pulsed Wave ultrasound apparatus which continually calculates velocity power spectra and displays a real time output of the velocity power spectrum so that its variation in time can be observed and analysed. The present invention additionally provides a real time volume flow rate associated with the observed region (i.e. the region defined by the receive beam widths and directions and the sample depth). Therefore when the clinician observes a pathological condition in the traditional power-velocity-time spectrum, he or she can immediately see an indication of the volume flow rate associated with that condition which can provide much easier, faster and more accurate diagnoses.

In preferred embodiments, a plurality of pulse-echo signals from spatially adjacent regions are acquired in parallel.

The parallel acquisition uses parallel beam forming to form a plurality of receive beams each directed at a different spatial region. Such parallel acquisition allows a sufficient spatial area to be covered within a small time frame. This is important in cases such as monitoring of live human or animal patients where organs may be moving in time, for example a beating heart. Restricting the time frame of acquisition, it is possible to acquire data from across the whole area of interest without significant movement of tissues within that area. The parallel acquisition may involve the simultaneous acquisition of data from all the spatially adjacent regions, but where this is not possible (e.g. due to beamforming or processing limitations), the group of spatially adjacent regions may be divided into a number of sub-groups, with the spatial regions within each sub-group being acquired in parallel and with the sub-groups being acquired in sequence.

According to a further aspect of the invention, which is believed to be independently inventive, there is provided a method of quantifying a fluid flow, the method comprising: transmitting a series of pulses, said pulses being transmitting at a series of pulse transmission times; acquiring a pulse-echo signal from each of a plurality of spatially adjacent regions; and processing said signals to form a velocity power spectrum; wherein the step of processing comprises: for each of a plurality of radial and lateral velocities, sampling more than one of said spatially adjacent pulse-echo signals at a series of sampling times, said sampling times being offset from said pulse transmission times by a variable offset which increases or decreases along the series according to the increasing or decreasing round-trip time from a potential scatter moving at said radial velocity, and wherein said samples are taken from the appropriate pulse-echo signal according to the expected lateral position of a potential scatterer moving at said lateral velocity.

Preferably the method further comprises: filtering said pulse-echo signals to exclude low frequency signal information. Preferably the method further comprises: adjusting said power spectrum to compensate for power lost in the filtering step, the adjustment being based on the calculated power spectrum and the characteristics of the filtering step.

According to a further aspect of the invention, there is provided an apparatus for quantifying a fluid flow, comprising: a transmitter arranged to transmit a series of pulses at a series of pulse transmission times; a receiver arranged to acquire a pulse-echo signal from each of a plurality of spatially adjacent regions; and a processor arranged to process said signals to form a velocity power spectrum; wherein the step of processing comprises: for each of a plurality of radial and lateral velocities, sampling more than one of said spatially adjacent pulse-echo signals at a series of sampling times, said sampling times being offset from said pulse transmission times by a variable offset which increases or decreases along the series according to the increasing or decreasing round-trip time from a potential scatter moving at said radial velocity, and wherein said samples are taken from the appropriate pulse-echo signal according to the expected lateral position of a potential scatterer moving at said lateral velocity.

Accordingly, velocities can be matched in both the radial direction (towards or away from the transmitter) and the lateral directions (perpendicular to the radial direction). This enables a better match of the velocities in the event that fluid flow is not perfectly aligned with the beam directions.

It will be appreciated that the preferred features described above in relation to the methods of displaying spatial data simultaneously with spatial data and the preferred features described in relation to the quantitative spectrum analysis without lateral velocity components can also be applied to the methods and apparatus of spectrum analysis incorporating lateral velocity tracking.

According to a further aspect of the invention, there is provided apparatus for quantifying a fluid flow, comprising: a transmitter arranged to transmit a series of pulses at a series of pulse transmission times; a receiver arranged to acquire a pulse-echo signal from each of a plurality of spatially adjacent regions; a filter arranged to exclude low frequency signal information from said pulse-echo signals; and a processor arranged such that for each of the pulse-echo signals, it: processes said signal to form a velocity power spectrum; and adjusts said power spectrum to compensate for power lost in the filtering step, the adjustment being based on the calculated power spectrum and the characteristics of the filtering step; wherein the step of processing comprises: for each of a plurality of velocities, sampling the pulse-echo signal at a series of sampling times, said sampling times being offset from said pulse transmission times by a variable offset which increases or decreases along the series according to the increasing or decreasing round-trip time from a potential scatterer moving at said velocity.

According to a further aspect of the invention, there is provided a software product, comprising instructions which, when carried out on a computer, cause the computer to: transmit a series of pulses, said pulses being transmitting at a series of pulse transmission times; acquire a pulse-echo signal from each of a plurality of spatially adjacent regions; filter said pulse-echo signals to exclude low frequency signal information; and for each of the pulse-echo signals, process said signal to form a velocity power spectrum; and adjust said power spectrum to compensate for power lost in the filtering step, the adjustment being based on the calculated power spectrum and the characteristics of the filtering step; wherein the step of processing comprises: for each of a plurality of velocities, sampling the pulse-echo signal at a series of sampling times, said sampling times being offset from said pulse transmission times by a variable offset which increases or decreases along the series according to the increasing or decreasing round-trip time from a potential scatterer moving at said velocity.

According to a further aspect of the invention, there is provided a method of manufacturing a software product which is in the form of a physical carrier, comprising storing on the data carrier instructions which when executed by a computer cause the computer to: transmit a series of pulses, said pulses being transmitting at a series of pulse transmission times; acquire a pulse-echo signal from each of a plurality of spatially adjacent regions; filter said pulse-echo signals to exclude low frequency signal information; and for each of the pulse-echo signals, process said signal to form a velocity power spectrum; and adjust said power spectrum to compensate for power lost in the filtering step, the adjustment being based on the calculated power spectrum and the characteristics of the filtering step; wherein the step of processing comprises: for each of a plurality of velocities, sampling the pulse-echo signal at a series of sampling times, said sampling times being offset from said pulse transmission times by a variable offset which increases or decreases along the series according to the increasing or decreasing round-trip time from a potential scatterer moving at said velocity.

According to a further aspect of the invention, there is provided a method of providing a software product to a remote location by means of transmitting data to a computer at that remote location, the data comprising instructions which when executed by the computer cause the computer to: transmit a series of pulses, said pulses being transmitting at a series of pulse transmission times; acquire a pulse-echo signal from each of a plurality of spatially adjacent regions; filter said pulse-echo signals to exclude low frequency signal information; and for each of the pulse-echo signals, process said signal to form a velocity power spectrum; and adjust said power spectrum to compensate for power lost in the filtering step, the adjustment being based on the calculated power spectrum and the characteristics of the filtering step; wherein the step of processing comprises: for each of a plurality of velocities, sampling the pulse-echo signal at a series of sampling times, said sampling times being offset from said pulse transmission times by a variable offset which increases or decreases along the series according to the increasing or decreasing round-trip time from a potential scatterer moving at said velocity.

The present invention extends to use of the above methods and corresponding apparatus in analysing blood flow in stenotic arteries, other blood vessels and/or heart valves. Preferably such use includes quantifying volume flow in such blood vessels or through such valves.

It will be appreciated that all preferred features described above in relation to the methods apply equally to the apparatus, uses and software.

It will also be appreciated that any or all features described above in relation to methods of displaying spatial data simultaneously with velocity data can also be applied with the methods of quantitative velocity spectrum analysis. The two concepts are highly related and strongly interlinked.

The inventions described above provide useful tools in obtaining and presenting information to a sonographer or clinician which may be used to facilitate diagnosis of various conditions.

Preferred embodiments of the invention will now be described, by way of example only, and with reference to the accompanying drawings in which:

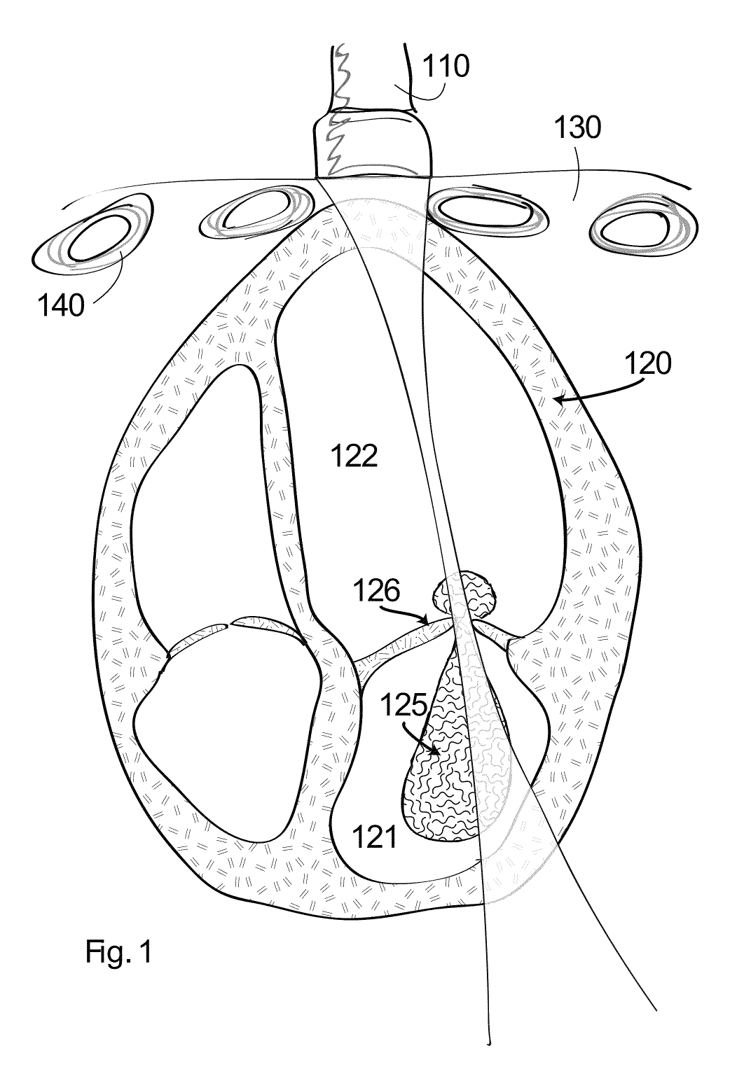

FIG. 1 illustrates a typical set up for ultrasound examination of a human heart;

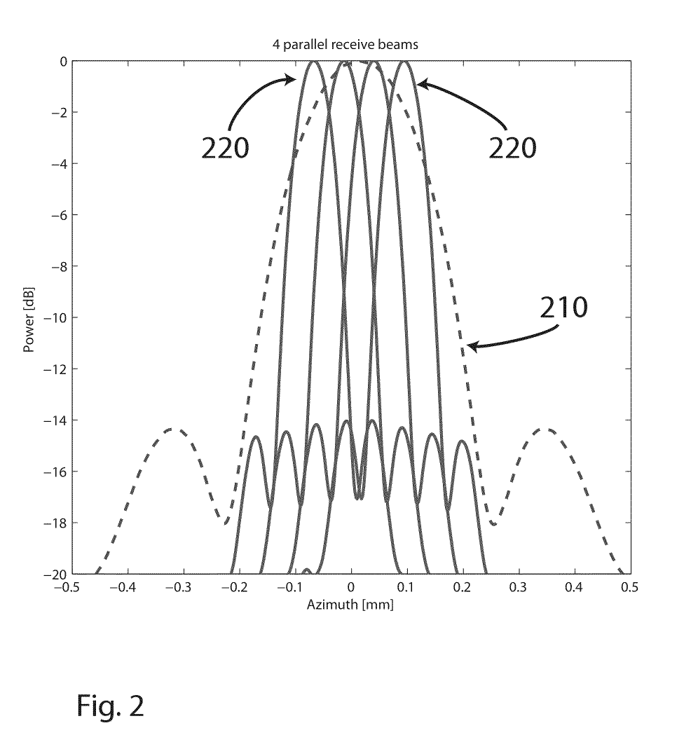

FIG. 2 illustrates parallel beamforming;

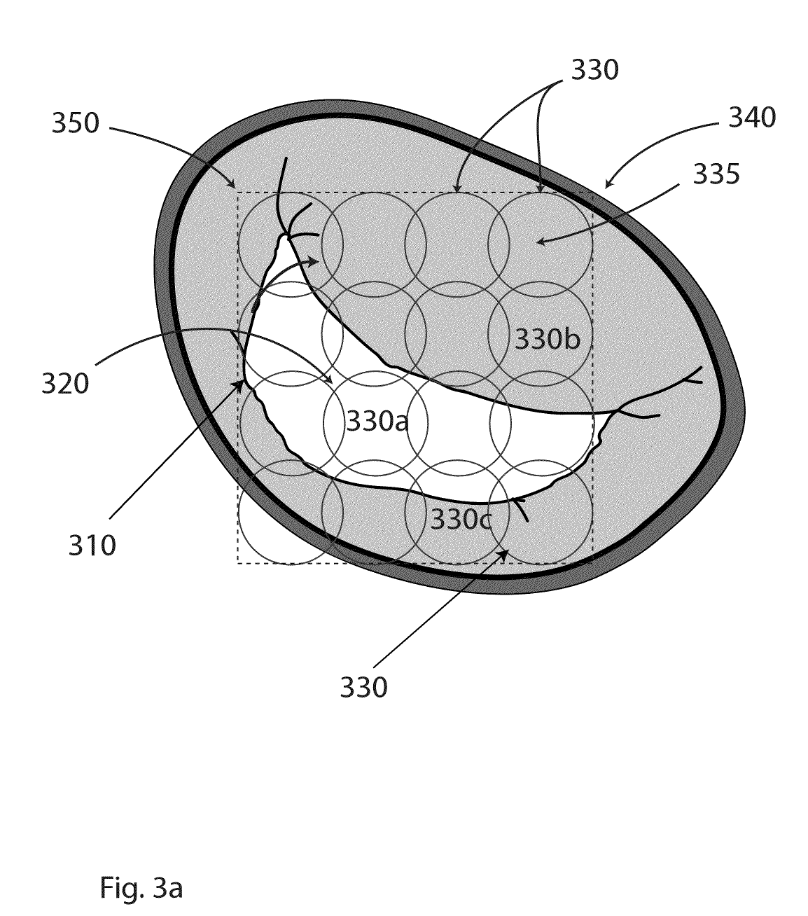

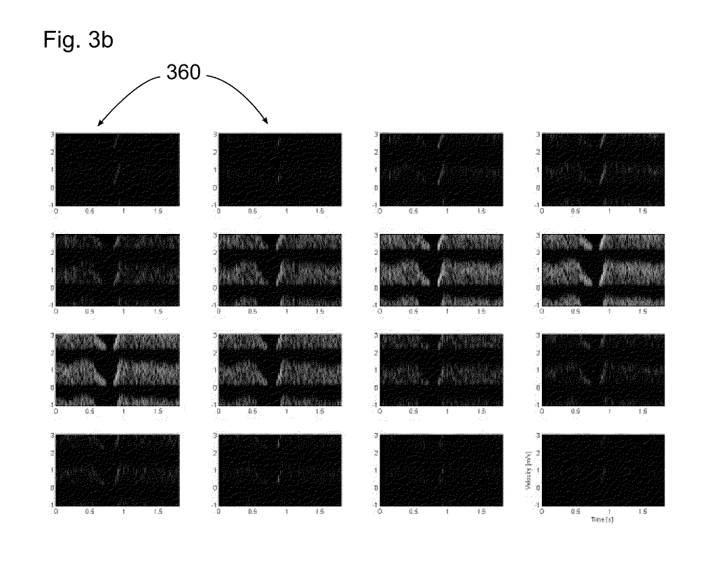

FIGS. 3a and 3b illustrates a leaky heart valve together with measurement beams;

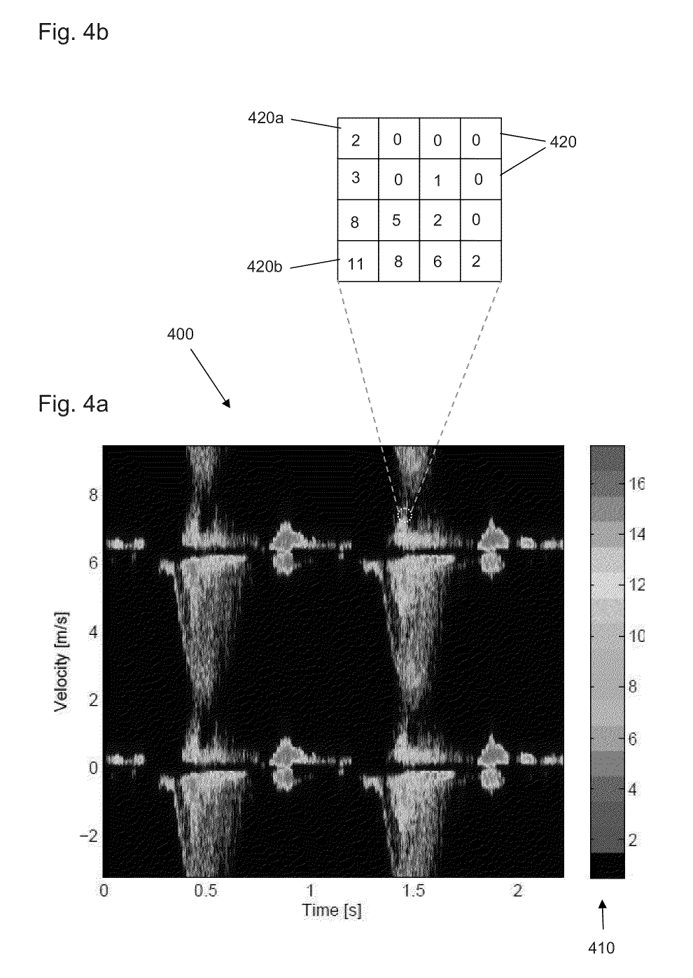

FIGS. 4a, 4b and 4c show a first method of displaying measured data;

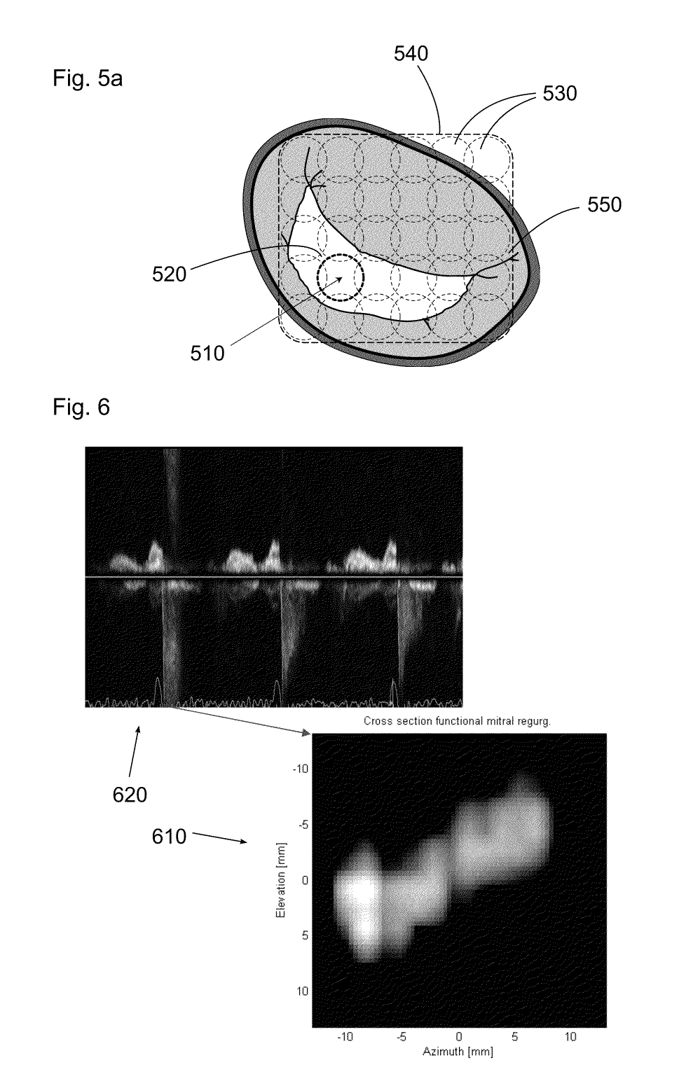

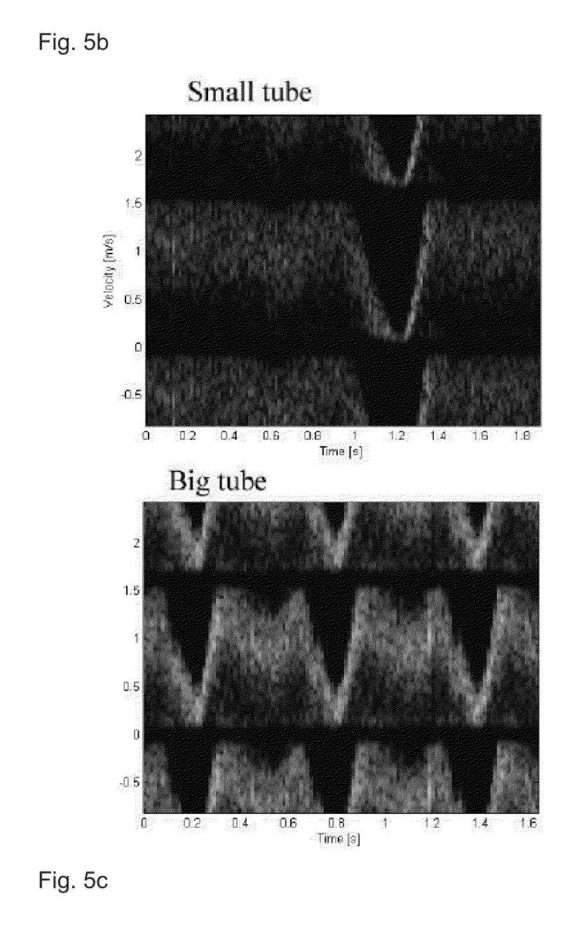

FIGS. 5a, 5b and 5c show a second method of displaying measured data;

FIG. 6 shows a third method of displaying measured data;

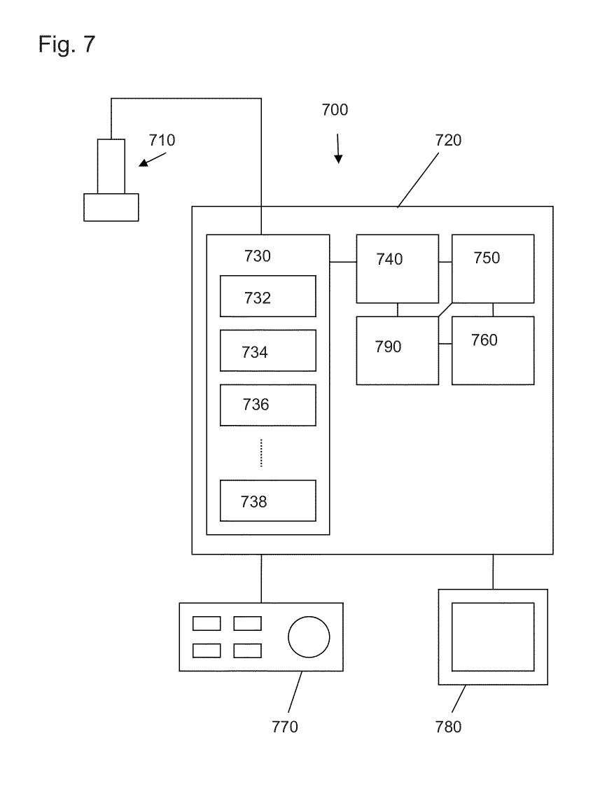

FIG. 7 schematically illustrates an apparatus for monitoring fluid flow.

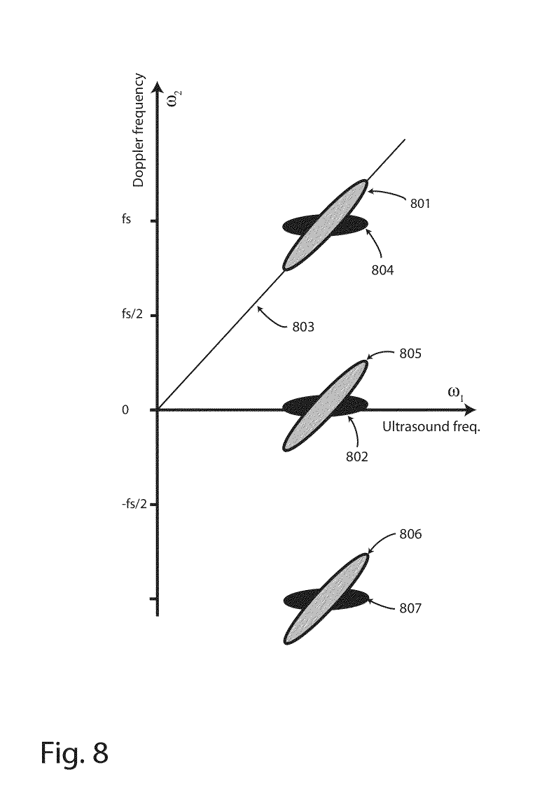

FIG. 8 shows a two dimensional fourier transform of an ultrasound signal;

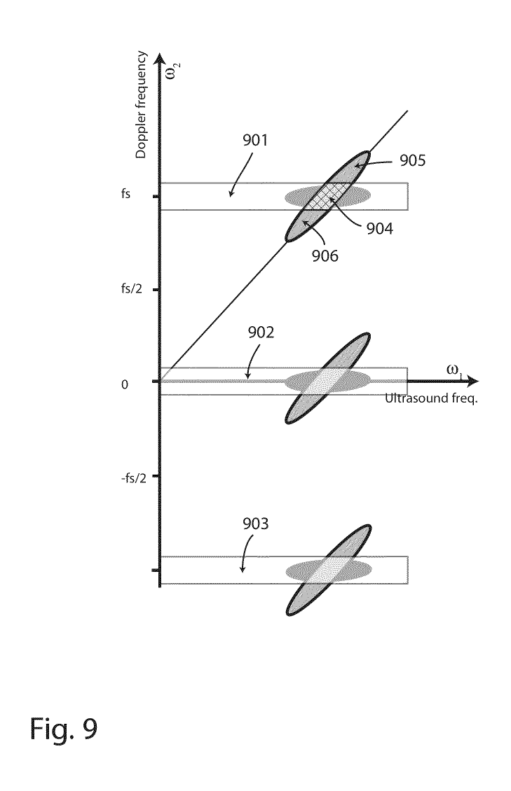

FIG. 9 is similar to FIG. 8, but also illustrates a clutter filter;

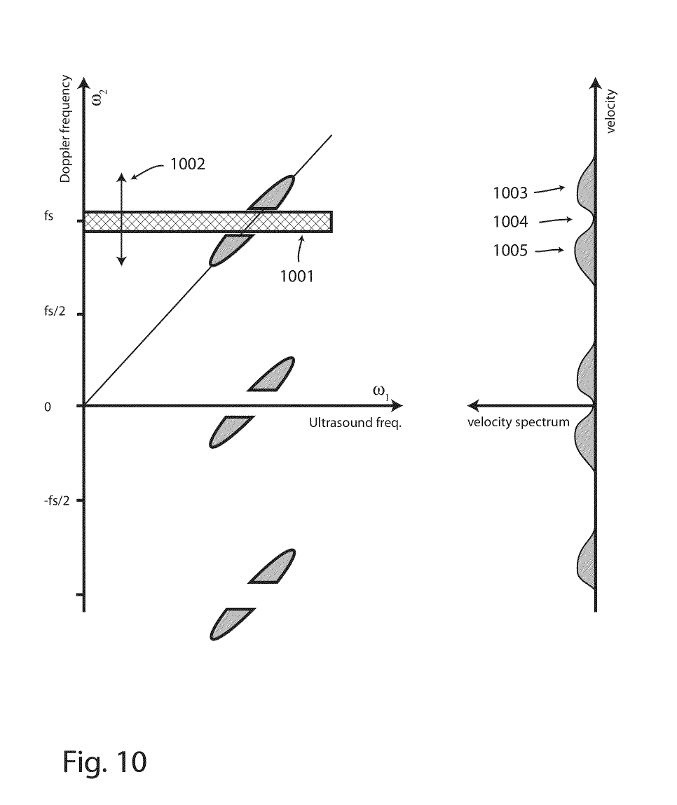

FIG. 10 shows the result of typical PW processing after applying the clutter filter in FIG. 9;

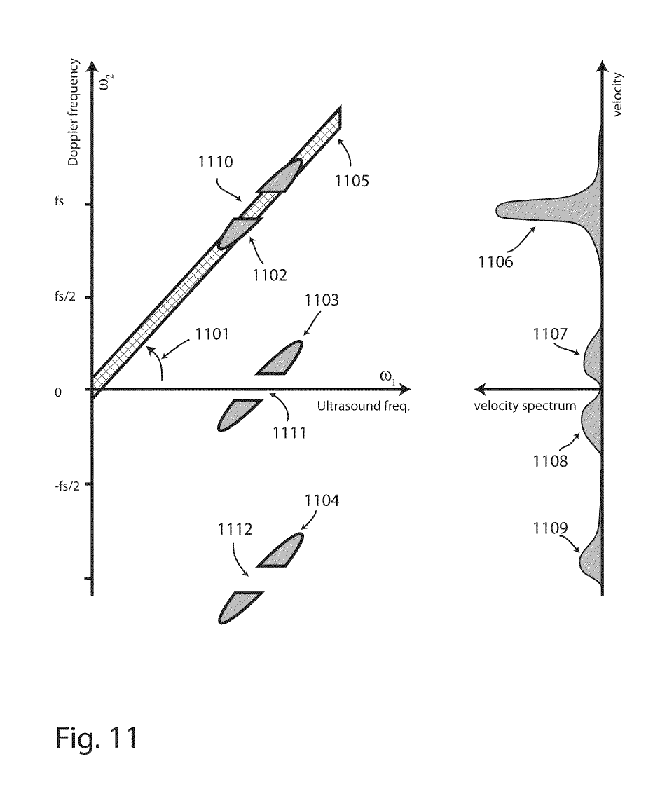

FIG. 11 illustrates the Velocity Matched Spectrum technique;

FIG. 12 illustrates the processing in the Velocity Matched Spectrum technique;

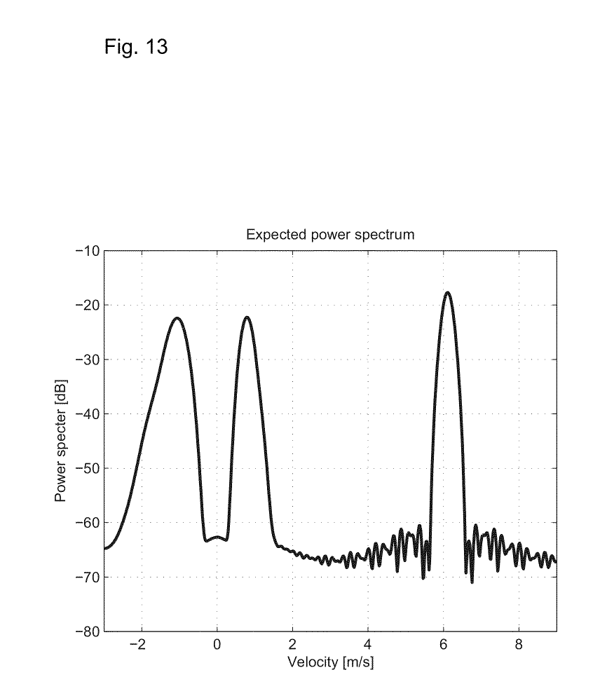

FIG. 13 shows an expected power spectrum from a simulation;

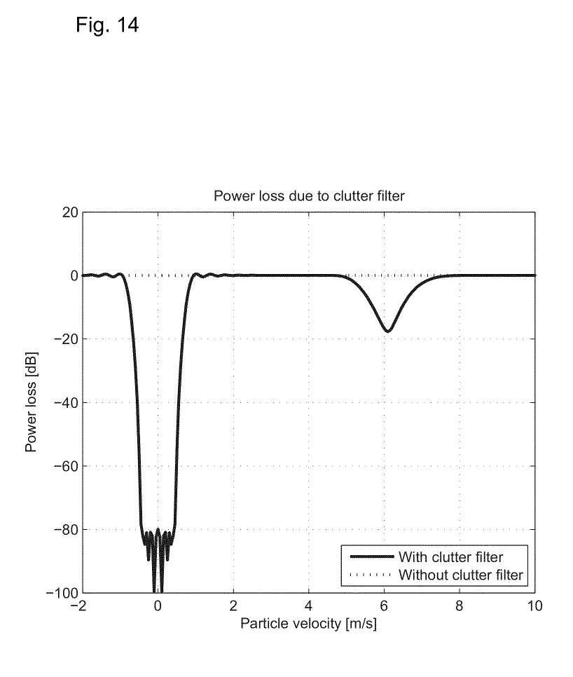

FIG. 14 illustrates the calculated power loss due to the clutter filter in the simulation of FIG. 13; and

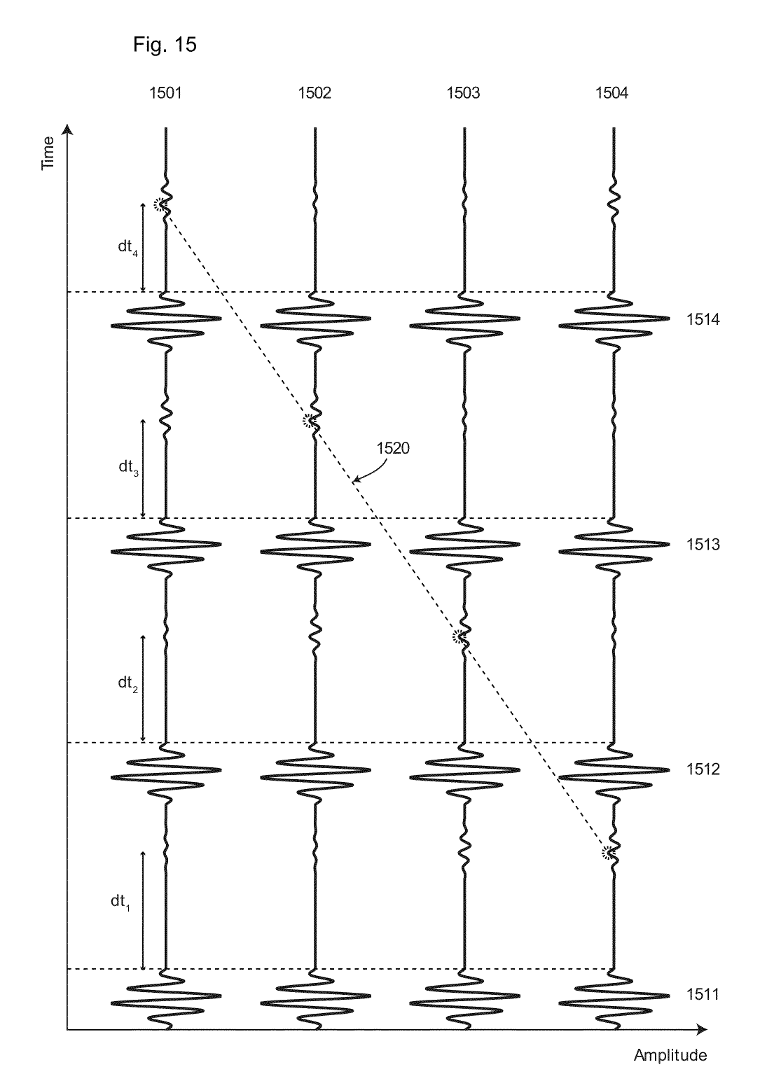

FIG. 15 illustrates a Velocity Matched Spectrum technique involving radial and lateral velocity tracking across multiple receive beams.

FIG. 1 illustrates a typical arrangement for monitoring a human heart. An ultrasound probe 110 is placed adjacent to the body 130 between ribs 140 and directed toward the heart 120. More specifically, the probe 110 is directed towards a mitral valve 126 which separates the left ventricle 122 from the left atrium 121. A mitral regurgitation jet 125 is shown with fluid flowing back through the mitral valve 126 in the wrong direction.

As will be discussed further below, the preferred embodiments of the invention make use of parallel receive beam forming techniques. Parallel beam forming is illustrated in FIG. 2 where a transmit beam 210 and a plurality of receive beams 220 are shown. As can be seen, the beam profile of the transmit beam 210 is wide enough to encompass four receive beams 220. The receive beams 220 can be formed and processed in parallel so that four different spatial regions can be processed from a single transmit beam. The advantage of this is that the pulse repetition frequency of the transmit pulses is a limiting factor on the speed of operation. Obtaining multiple receive signals from a single transmit beam means that a given area can be interrogated at a given resolution faster than if each receive beam required its own transmit beam.

The ratio of receive beams to transmit beams can vary depending on the equipment available and the type of measurements being performed. It is necessary to bear in mind that the width of the transmit beam cannot be increased indefinitely as widening the transmit beam reduces the energy density and in turn the signal-to-noise ratio (SNR). This puts restrictions on the number of parallel beams that can practically be obtained from a given transmit beam. Also, the profile of the transmit beam is not constant and therefore sensitivity varies across the beam, which means that the received gain will vary depending on the position within the transmitted beam. In some preferred embodiments this can be compensated for by adjusting the gain of the individual receive beams. In some preferred embodiments four receive beams are used to obtain receive signals from each transmit pulse. In other embodiments, sixteen receive beams are used to obtain receive signals from each transmit pulse. For example a grid of four by four receive beams can be formed and the signals processed simultaneously. These arrangements are utilized in some preferred embodiments, but it should be understood that, in principle, any number of receive beams can be used. It is also possible to use a combination of parallel and sequential beam forming. For example, a set of sixteen regions could be divided into four subsets of four beams, with the four beams of each subset being obtained in parallel and the four subsets being obtained in sequence.

The arrangement of transmit beams and receive beams can be varied according to the particular situation being examined. FIG. 3a illustrates a heart valve 310 in the closed state with an orifice 320 which is formed because the valve 310 does not close properly. The orifice 320 results in a regurgitation jet such as the one illustrated at 126 in FIG. 1 (in the case of a mitral valve). FIG. 3a also shows a matrix 340 of receive beams 330 directed at and around the orifice 320. All together, the receive beams 330 of the matrix 340 cover the entire area of the orifice 320. Some receive beams 330 will fall completely within the orifice area (e.g. 330a), some will fall completely outside the orifice area (e.g. 330b) and some will straddle the boundary of the orifice 320 (e.g. 330c). Depending on the size of the orifice, more or fewer beams may be required to cover the whole orifice area. The matrix 340 may be a linear array of receive beams (in which case a linear or phased array transducer will suffice for obtaining the measurements), but is preferably a two dimensional array of receive beams (in which case a two-dimensional transducer array is required for beam steering).

Each beam 330 is directed at an individual region 335 within the measurement area 350 covered by the matrix 340. For each region 335, a power-velocity spectrum 360 is obtained by Pulsed Wave Doppler techniques. With Pulsed Wave ultrasound, the measurement area 350 illustrated in FIG. 3 is in fact a thin measurement volume within the patient. The depth of the volume within the patient is selected by range gating of the received echo signal. For measurement of a heart valve as illustrated, the volume 350 is typically selected to be in the vena contracta of the regurgitant jet, i.e. just behind the orifice.

The power-velocity spectrum is typically obtained by known techniques based on a series of many successive transmit pulses (typically between 60 and 100 pulses per time interval) resulting in a corresponding series of received echo signals. The power-velocity spectrum is generated repeatedly in time so as to illustrate the changes in power and velocity with time within the volume 350. The echo signals may be stored in memory and thus the series of successive echo signals used to form the power-velocity spectrum may be selected according to a sliding time window (i.e. when a new signal is received it is added to the series, while the oldest signal is dropped from the series).

The power-velocity spectra obtained from the plurality of spatial regions 335 together form a data set containing both spatial information about the orifice and velocity information about fluid flow through the orifice. Both the spatial data and the velocity data provide useful information about the orifice under examination. One option for displaying this information to the sonographer or the clinician is to display the full spectrum for each individual region as illustrated in FIG. 3b. In FIGS. 3a and 3b, sixteen receive beams are used to interrogate the orifice and its surroundings. Sixteen spectra corresponding to each of the sixteen individual regions are displayed simultaneously. It can be seen that the spectra corresponding to the regions within or partly within the orifice show greater power at greater velocities than the spectra for the regions which fall outside the orifice. The data thus present information not only about the velocities within the orifice, but also spatial (geometrical) information about the shape of the orifice.

The full spectra display illustrated in FIG. 3b presents a significant amount of information all at once. This can be a useful display of the combined data set when the user has enough time to examine and evaluate all of the data presented. This will often be the case when the data set is recorded and subsequently evaluated some time after the sonographic examination took place. However there is also a need to provide a display of the combined spatial/velocity data set in real time during the examination itself. This provides the sonographer with real time feedback about both the spatial information and the velocity information. This information combined provides a good indicator of the size and/or severity of the orifice. When the sonographer is able to see this information in real time, he or she can assess the need for alternative or more detailed views to be obtained or for further examinations to be conducted immediately so that a better diagnosis can be made later. This can reduce the likelihood of having to recall the patient for further tests at a later time.

Three methods of presenting the combined data set with aspects of both the spatial data and the velocity data are presented in FIGS. 4a and b, 5a, b and c and 6. The first two methods provide a predominantly velocity based view in the form of a velocity spectrum with spatial information added thereto. The third method provides a predominantly spatial view with velocity information added thereto.

FIG. 4a shows a visual display 400 similar to that of a standard power-velocity spectrum display on an ultrasound unit, but with additional spatial information added in the form of colour.

The power-velocity spectrum display of standard ultrasound units shows time along the horizontal axis and velocity along the vertical axis. For each time step, a power-velocity spectrum is displayed vertically at the corresponding position on the time axis. The velocities in the spectrum are typically divided into a number of discrete velocity bins (i.e. velocity ranges) and each bin is displayed at the appropriate position along the velocity axis with a brightness determined by the received signal power associated with the velocities in that bin. This is typically accomplished on an electronic display by illuminating one or more pixels an appropriate amount.

In the display shown in FIG. 4a, the colour of each velocity bin is determined by the number of spatial regions 335 for which the power in that velocity bin is above a certain threshold. In other words, for each velocity bin, the power in the corresponding velocity bin of each of the individual region spectra is compared against a threshold and the number of spectra which are above that threshold is used to determine the colour of the composite display.

As an example, taking the sixteen individual spectra shown in FIG. 3b, a velocity bin corresponding to a high velocity will contain significant power only if the individual region lies within a regurgitant jet. Therefore in the case of a small orifice, only one or two regions will contain power above the threshold in that velocity bin, whereas in the case of a large orifice, there may be 6 or 7 (or more) regions with power above the threshold in that velocity bin. This spatial information in the form of the number of regions above threshold can be displayed on the composite display output in the form of a colour. For example, the scale 410 shown in FIG. 4a shows the number of regions from 0 up to 16, with colours starting from black at 0, passing through progressively lighter blues from 2 up to 6, then greens up to 10, yellow at 12, orange at 14 and red at 16. These colours are easily and immediately recognizable by the sonographer. A velocity bin coloured red on the output indicates that all of the individual spatial regions 335 are showing high power at that velocity, whereas a velocity bin coloured dark blue on the output indicates that only 2 individual spatial regions are showing high power at that velocity. This immediately distinguishes a large jet (and hence a large orifice) from a small jet (and hence a small orifice). The display is particularly convenient for the sonographer because it is in the general form of a standard spectral output with which he or she will already be extremely familiar. The added information is therefore easy to recognise and interpret in a familiar context.

An appropriate threshold will of course need to be determined, but that will depend on the circumstances such as the type of examination being performed and the typical velocities and power to be encountered. The threshold may be automatically determined in software or hardware based on the sensed data or it may be selected by the user either on a continuous or discrete scale or it may be selected from a number of stored preset values.

FIG. 4b shows a large scale illustration of a number of velocity bins 420 as depicted in a typical spectral plot, i.e. with time along the horizontal axis and velocity along the vertical axis. Brightness data (i.e. power data) is not illustrated in FIG. 4b, but the spatial data is indicated by the numbers shown within the velocity bins 420. The numbers here indicate the number of individual regions from the overall measurement which have a power above the threshold value. For instance, the velocity bin 420a in the upper left corner of FIG. 4b contains the value ‘2’ indicating that two of the spectra (i.e. two of the individual spatial regions) contained power above the chosen threshold in the velocity range corresponding to that bin 420a at the point in time indicated by the leftmost column in FIG. 4b. Similarly, the velocity bin 420b in the lower left corner of FIG. 4b contains the value ‘11’ indicating that eleven of the spectra (i.e. eleven of the individual spatial regions) contained power above the chosen threshold in the velocity range corresponding to that bin 420b at the point in time indicated by the leftmost column in FIG. 4b. The value of ‘11’ in bin 420b indicates that the velocities corresponding to bin 420b were strongly represented at that point in time across a large spatial area.

FIGS. 5a, 5b and 5c show an alternative composite display output for the combined spatial and velocity data set. A heart valve 550 is depicted in FIG. 5a similarly to the valve 310 depicted in FIG. 3a, with a plurality of spatially adjacent measurement beams 530 directed at and around the orifice. One of the beams 510 has been identified as a reference beam.

For this display method, the spatial data are divided into two groups. In this example, the first group consists of just the reference beam 510 and the second group consists of any number N of the received beams 530, for instance all of them (or all except the reference beam 510). The idea with this method is to compare the power of the first group (the reference beam 510) to the power of the second group (the rest of the measurement beams 530). The power of the second group is weighted to have the same weight as the first group, i.e. for N total beams with one reference beam the power of the second group (of N−1 beams) is given a weighting factor of 1/(N−1).

A first colour is allocated to the first group and a second colour is allocated to the second group. In this embodiment depicted in FIGS. 5a, 5b and 5c, the second colour (cyan) is complementary to the first colour (red), i.e. when the two colours are combined together in equal measure they produce a shade of grey. If the signal power is maximum relative to the dynamic range of the display the spectrum is shown as white. Similarly, if the power is minimum the spectrum will be black, and any value in between will be a shade of grey.

The reference beam 510 in this embodiment is selected dynamically to be the beam with the highest overall power as this is likely to be a beam falling wholly within the orifice 520.

The display output is formed by combining (for each velocity bin) the power of the first group with the power of the second group, by adding an amount of the first colour corresponding to the power in that velocity bin of the first group to an amount of the second colour corresponding to the power in that velocity bin of the second group. The output display colour for each velocity bin is therefore on a sliding scale from the first colour to a shade of grey, with a whiter colour indicating a larger orifice. For example, in the embodiment described here, if the reference beam is the only beam falling within the orifice (i.e. if the orifice is small) and all other measurement beams fall outside the orifice, the combined colour will be red (the first colour) as the contribution from the second group is negligible. This situation is illustrated in FIG. 5b. On the other hand, if the orifice is very large, then all (or nearly all) of the measurement beams could fall within the orifice. In that situation, the weighted power of the N−1 non-reference beams 530 may substantially equal the power of the reference beam 510. The result is that the output colour of the composite display is produced from substantially equal amounts of red and cyan and is therefore a shade of grey. FIG. 5c illustrates a situation for a flow which is large compared with that of FIG. 5b. The flow clearly does not encompass all beams 530 as the display is not grey, but the displayed spectrum is a much lighter colour than that of FIG. 5b and therefore indicates a much greater contribution of cyan to the overall colour. In turn this indicates a larger power contribution from outside the reference beam 510.

In a similar way to the first display method, this method provides an output display in the familiar form of a spectral display, but with added colour which is derived from the spatial information in the combined data set and which indicates the size and/or severity of the orifice. The information is displayed so that it can be quickly and easily interpreted in the familiar context.

In a related embodiment the reference beam 510 is not selected dynamically, but is fixed as one specific beam, for instance the beam closest to the centre of the array of beams 540. If the reference beam 510 does not fall within the orifice 520, but some of the other beams do, the spectrum will be coloured in a shade of the second/complementary colour (cyan in the previous example). In this embodiment the operator can use the colour of the display to fine-tune the position of the probe, knowing that when the spectrum colour turns a shade of the first colour (red) or grey, then the array of beams 540 is centred around the jet (and hence the orifice 520).

In other modifications of this embodiment, the first and second groups of receive beams need not be complementary (i.e. not all receive beams must be used). Likewise, the first group need not be a single beam, but could be a plurality of beams. The beams of the first group are preferably adjacent to each other, but need not be. In yet further modifications, there may be three or more groups, each with a different colour associated with it. This technique might be useful in situations where multiple orifices exist. In one example, three groups can be identified, and each group associated with a primary colour, i.e. one of red, green and blue. The overall combined colour can then readily be used to identify the relative strengths of each of the three groups. As before, the signals from the groups may be weighted according to the size (number of beams) of the group.

FIG. 6 illustrates a third method of displaying both spatial and velocity aspects of the combined data set. This method differs from the first two methods in that it provides a predominantly spatial view of the data with velocity information added to it, whereas the other two methods display predominantly velocity information with spatial information added.

The display illustrated in FIG. 6 shows a spatial plot 610 of the total received signal power in each individual region (335, 530) at a particular point in time (the particular point in time is indicated on the accompanying spectral plot 620). The plot 610 shown in FIG. 6 also shows interpolated points derived from combinations of the raw spectral data. The overall shape of the plot indicates the overall geometry (and hence the size and shape) of the orifice. For each spatial point on the display, the colour is determined according to the velocity data in the corresponding individual region spectrum (or for an interpolated point, according to an interpolation of velocity data from neighbouring individual regions).

The particular colouring technique illustrated here determines the colour from the maximum velocity for that spatial region. Colour may be selected based on a continuous velocity scale or based on discrete velocity ranges. For example the technique illustrated here divides the range of velocities into a low range (coloured green), a mid range (coloured yellow) and a high range (coloured red). This provides an immediate indication to the sonographer of both the size of the orifice and the velocities of flow through the orifice. This enables a rapid and easy identification of, for example, a small orifice with low flow, a large orifice with low flow and a large orifice with high flow. This geometrical display is also particularly useful in that it can identify multiple distinct jets within the measurement region.

Another way to determine the colours of this display is to realize that the data set is a 3D dataset, where two dimensions are spatial and the third dimension is Doppler frequency/velocity. When this data set is viewed from above, looking along the third dimension, the result is the display in FIG. 6. If a number of different colours are assigned, say a colourmap with 64 colours, along the velocity axis, the colour when viewed from above can be determined using regular volume rendering techniques known in computer graphics, incorporating the power of the signal at each velocity bin to determine how transparent/opaque each voxel should be. In this way a high-power, high-velocity jet would be coloured according to the high velocity, but a low-power, high-velocity jet will be transparent and show the colours of the velocities beneath. The 3D data set, when displayed in this way, can also be visualized from other angles, for instance from the side.

It will be appreciated that the embodiments described in FIGS. 4a,b 5a-c and 6 all represent the same underlying composite data set. Therefore all of these display techniques can be used alongside each other to provide different representations to the operator. These display techniques may be displayed simultaneously or may be selectable by the user. In certain preferred implementations, at least one of the spectral displays is displayed simultaneously with the geometrical display.

FIG. 7 schematically illustrates an apparatus for implementing the above-described methods. The system 700 includes an ultrasound probe 710 (preferably a matrix-array-type probe) connected to a computer 720. The computer 720 includes a beamformer 730 which is connected to a signal processor 740 which in turn is connected to a CPU 750. Beamformer 730 comprises a transmit beamformer 732 and a plurality of receive beamformers 734, 736, 738 which can form parallel receive beams for simultaneous data acquisition. A memory 760 is also provided. Memory 760 may include one or both of permanent memory (e.g. hard disks or flash memory) and temporary memory (e.g. random access memory). Computer 720 also includes a GPU 790 which can be used for additional processing, particularly graphical based processing. An input panel 770 (which may include one or more of keyboard, mouse, trackball, joystick or other input controls) is also connected to the computer 720 to enable operator input to the apparatus 700 and control of the apparatus 700. A display 780 is also connected to the computer 720 for displaying the output of the apparatus 700.

It will be appreciated that FIG. 7 is very schematic in nature and that it represents only one possible configuration of equipment. Further features such as motherboards, buses, other peripherals (e.g. printers, speakers, optical drives) may be provided as required. Similarly it will be appreciated that the various processing aspects of the invention may be implemented on different parts of the system. For example, the processing performed by the beamformer 730, signal processor 740 and CPU 750 may all be performed by a single device or may be distributed among any number of processors. In particular, it should be noted that processing is not restricted to within the computer 720, but some or all processing may be performed within the hand-held probe 710. The processing required of the system may be carried out on one processor or may be shared between several processors. These processors may include one or more CPUs and/or one or more GPUs.

To be able to perform quantitative Doppler measurements using the techniques of the present invention, it is necessary to estimate the power of the Doppler signal from the fluid (e.g. blood). This can be done in several ways. Two of the most common ways are:

1) by estimating the power-velocity spectrum using Welch's method (the modified periodogram method) and extracting the Doppler power from the velocities of interest (see for example U.S. Pat. No. 6,544,181), or
2) by estimating the zero'th lag of the autocorrelation-function typically after removing the signal from low-velocity scatterers by a clutter filter (see for example US 2009/0043208).

Both of these methods will be unable to accurately estimate the Doppler power if the blood is travelling at a radial velocity of approximately two times the Nyquist velocity. This is because the clutter filter will remove a band close to each multiple of the sampling frequency, which means it will also remove the Doppler signal. At velocities exceeding two times the Nyquist velocity the transit time broadening will increase the spectral bandwidth which makes it hard to separate the aliased signal from non-aliased parts of the signal.

A method for generating power-velocity spectra with suppressed velocity ambiguity is discussed in U.S. Pat. No. 5,662,115. This technique is known as the “Velocity Matched Spectrum” technique. It works by tracking the scatterers in the two-dimensional range/time data set. In practice this is done by integrating along skewed lines in the range/time plane, where the slope is chosen to follow the movement of the scatterers along the ultrasonic beam for each velocity in the spectrum. When the slope matches the actual velocity, the received echoes will match both in phase and amplitude, giving a peak in the spectrum at the actual velocity value. This can be seen in FIG. 12, where integration along the line in the range-time plane corresponding to the actual velocity leading to a peak in the spectrum, while integrating along the two lines which do not correspond to the actual velocity does not.

By the Fourier projection-slice theorem the process can be performed and explained in the two-dimensional frequency domain instead of in the depth/time domain. By integrating the 2D power spectrum along lines through the origin for a number of predefined velocity values, a velocity spectrum with suppressed aliasing can be constructed. Fourier analysis makes it easier to understand the benefits of the velocity matched spectrum tracking for this application.

A two-dimensional Fourier transform of the received signal, from several ranges and pulses, will give a power spectrum similar to the example shown in FIG. 8. The horizontal axis is the transmitted frequency, and the vertical axis is the Doppler frequency shift. fs/2 is the Nyquist limit, which corresponds to the Nyquist velocity

$${v}_{n} = \frac{cPRF}{4 f} ,$$

where c is the speed of sound, PRF is the pulse repetition frequency and f is the center frequency of the pulse. The two-dimensional power spectrum shows how each frequency component of the transmitted signal creates a Doppler shift 801 proportional to the velocity and the transmitted frequency, as stated by the Doppler equation. This diagonal 803 is indicated in the Figure. In this example the blood velocity is approximately the same as fs such that the Doppler signal and the clutter signal 802 overlap. Since the clutter is independent of the transmitted frequency of the probe, it does not follow the iso-velocity lines, but contributes equally at all ultrasound frequencies. Since these are sampled signals both the Doppler signal 801 and the clutter signal 802 will alias/repeat at each multiple of two times the Nyquist velocity. This sketch shows the aliased Doppler signal 805, 806 and the aliased clutter signal 804, 807.

When applying a clutter filter as shown in FIG. 9, the part 904 of the Doppler signal which corresponds to the clutter filter 901, 902, 903 bandwidth is removed together with the clutter 804. This portion of the signal 904 is indicated with a checkered pattern, and losing this part of the spectrum means that it is no longer possible to accurately estimate the Doppler power. When the pulse bandwidth is sufficiently large, parts 905, 906 of the Doppler signal will still be intact outside the cutoff band of the filter. Note that the clutter filter stop band also repeats every multiple of fs.

FIG. 10 shows the window 1001 used for traditional power spectrum estimation after clutter filtering. There is little to no overlap between the remaining Doppler signal inside the clutter filter stop band and the window, meaning that the Welch spectrum estimator will fail to detect the highest power region of blood flow which should have been present in the spectrum at 1004. To estimate the full velocity spectrum the window 1001 is moved linearly along the Doppler frequency axis, as indicated by the arrow 1002. There may be smeared out parts 1003, 1005 of the velocity spectrum around where the peak should have been 1004. Since it is a sampled signal this conventional Doppler spectrum method will repeat the same pattern at each multiple of two times the Nyquist frequency in the spectrum.

However, if the window 1105 is positioned at an angle 1101 as shown in FIG. 11, as in the Velocity Matched Spectrum technique, the window will contain most of the remaining Doppler power when the angle matches the angle given by the Doppler equation, and give a peak 1106 in the velocity-matched power spectrum. The intensity of the peak will be reduced due to the part of the Doppler spectrum filtered out by the clutter filter, but this has been of little importance for the applications of the prior art, as the peak velocity can typically be accurately delineated, e.g. on a visual display despite the reduced intensity of the peak. However, for quantitative purposes the Doppler power needs to be estimated more accurately.

In contrast to the traditional processing where the spectrum is repeated every 2vₙ, the velocity matched spectrum method favors the velocities following the iso-velocity lines, and thereby suppresses the copies at the Nyquist frequencies. The aliased versions of the signal will result in lower-amplitude, smeared out elements 1107, 1108, 1109 in the spectrum as shown in the right pane of the figure.

The following description explains how preferred embodiments of the invention make use of the Velocity Matched Spectrum method together with compensation for the effect of the clutter filter in order to produce quantitatively accurate velocity spectra which can be used to quantify fluid flows.

FIG. 8 shows a two dimensional fourier transform of an ultrasound signal from several ranges and several pulses, containing both a clutter signal 802 and a Doppler signal 801 from stationary flow. Since the signals are sampled they repeat at two times the Nyquist velocity, producing aliased versions of the clutter signal 804, 807 and the Doppler signal 805, 806. The moving scatterers creates a Doppler shift 801 proportional to the velocity and the transmitted frequency, as stated by the Doppler equation and indicated by 803.

FIG. 9 shows the same situation as FIG. 8, but including the stop-band of a clutter filter 902, also repeated at multiples of two times the Nyquist frequency 901, 903. The Doppler power is shown split into three regions 904, 905, 906 where 904 is lost to the clutter filter.

FIG. 10 shows the remaining signal after application of the clutter filter. A window 1001 moving along the Doppler frequency axis 1002 is shown. The window 1001 represents the conventional technique for obtaining a velocity spectrum and selects the signal used to obtain the velocity spectrum shown to the right. 1003, 1004 and 1005 indicates the spectrum resulting from the Doppler shifts in 905, 904 and 906, respectively, where 1004 the peak is missing due to clutter filtering.

FIG. 11 shows the situation when using Velocity Matched estimation. The window 1105 is rotating 1101 around the origin selecting the frequency contents it overlaps 1102. A peak in the spectrum 1106 is formed when the angle of inclination matches the actual scatterer velocity. When the window overlaps aliased signals 1103, 1104 it produces smeared out parts of the spectrum 1107, 1108, 1109. Parts 1110, 1111, 1112 of the 2D spectrum are still missing after clutter filtering, but the shape of the velocity spectrum can still be reconstructed, albeit with lower power.

FIG. 12 shows an illustration of the received echo signals from several pulses transmitted towards scatterers moving at a given velocity. The right part of the figure illustrates the power spectrum which will result from integrating along diagonal lines in the range-time plane in the left part of the figure. Integrating along the line in the range-time plane corresponding to the actual velocity leads to a peak in the spectrum, while integrating along the two lines which does not correspond to the actual velocity does not.

FIG. 13 shows a simulation of the expected power spectrum when the scatterers are moving at 6.1 m/s, which is two times the Nyquist velocity for the particular system settings used in the simulation. Note that there is a peak in the spectrum corresponding to the actual velocity.

FIG. 14 is a simulation showing how the peak value of the power spectrum (like the one in FIG. 13) varies with scatterer velocity. Note the loss of power when the velocity is close to two times the Nyquist velocity (6.1 m/s). Knowledge of this power loss is used to compensate for the effect of the clutter filter in the power estimate.

### Signal Model

In the following, it will be shown how the Doppler power can be estimated by taking the known frequency response of the clutter filter into account. To do this we must first introduce a signal model.

The received complex demodulated signal (the IQ signal) x(t,k), where t corresponds to a depth range r=(c/2)t and k is the pulse transmission, is a complex Gaussian process. The RF signal is the real part of the complex pre-envelope of the signal which can be expressed as

xₚ(t,k)=x(t,k)e^{iω}^{₀}^{t}. (0.1)

The autocorrelation function of the pre-envelope signal is given by

Rₓ_{ₚ}(τ,m)≡ [symbol] xₚ*(t,k)xₚ(t+τ,k+m) [symbol] , (0.2)

where [symbol] [symbol] is the expected value.

The power spectrum is the 2D Fourier transform of the autocorrelation function

$$G ( {ω}_{1} , {ω}_{2} ) ≡ {\sum }_{m}^{ } \int d {τR}_{{x}_{p}} ( τ , m ) {e}^{i {ω}_{1} τ} {e}^{{iω}_{2} mT} , \qquad ( 0.3 )$$

where ω₁ and ω₂ are the angular frequencies in fast and slow time.

We look at a situation with a flow containing the radial velocity v₀. Change in arrival time from pulse to pulse caused by v₀ is given by

$${δ}_{{v}_{0}} = - \frac{2 {v}_{0} T}{c} , \qquad ( 0.4 )$$

where T is the pulse repetition period. Positive direction for the velocity is towards the probe. We want to calculate the power from all possible radial velocity components v. The change in arrival time from pulse to pulse caused by v is given by δ=−2vT/c.

By tracking each potential scatterer and summing up the power received from each of them the velocity spectrum can be calculated

$$\hat{p} ( v ) = {\vert {\sum }_{k}^{ } w ( k ) {x}_{p} ( {t}_{0} + k δ , {k}_{0} + k ) \vert}^{2} \qquad ( 0.5 )$$

where w(k) is a rectangular or smooth window function applied to the data, for instance a hamming window to reduce sidelobes in the spectral estimate.

Assuming a stationary and uniform velocity field the autocorrelation function can be written as

Rₓ_{ₚ}(τ,m;v₀,v₁)=s₂(τ−mδᵥ_{ᵣ})b₂(vₜTm) (0.6)

where s(t) is the point echo response from blood and b is the beam sensitivity function which is constant when the transversal velocity component vₜ is zero. Subscripts s₂ and b₂ are short notation for the autocorrelation operator applied to s and b, respectively; T is the pulse repetition period, and c the speed of sound.

The power spectrum can be written as a sum of copies of the non-aliased part of the signal, G₀(ω₁, ω₂), with distances equal to the angular pulse repetition frequency 2π/T, and where G₀(ω₁, ω₂) is given by

G₀(ω₁,ω₂)≡∫dm∫dτRₓ_{ₚ}(τ,m)e^{iω}^{₁}^{τ}e^{iω}^{₂}^{T}. (0.7)

By combining equation (0.6) and (0.7) the power spectrum can be written as

$$\begin{matrix}G ( {ω}_{1} , {ω}_{2} ) = {\sum }_{n}^{ } {G}_{0} ( {ω}_{1} , {ω}_{2} + n \frac{2 π}{T} ) \\ = {\sum }_{n}^{ } {\vert S ( {ω}_{1} ) \vert}^{2} {\vert B ( \frac{1}{{v}_{t}} ( {ω}_{2} - \frac{2 v}{c} {ω}_{1} + 2 n π ) ) \vert}^{2} ( 0.9 )\end{matrix} \qquad ( 0.8 )$$

where S(ω₁) and B(ω₂) are the Fourier transforms of s and b, respectively. The frequency response S(ω₁) limits the spectral bandwidth in the ω₁ direction. For n=0 the argument of B is zero along a straight line through the origin, which represents the Doppler equation. This line is drawn in the Frequency-domain figures. Since B is a lowpass function the spectral energy is concentrated along this line, and the bandwidth in the ω₂ direction is proportional to the lateral velocity component. In a general non-uniform velocity field the signal power spectrum can be obtained by integrating the spectrum in (0.8) over the velocity distribution inside the ultrasonic beam, that is, by integrating along curves instead of straight lines.

Now let us apply a clutter filter, h(k), to the received signals:

yₚ(t,k)=xₚ(t,k) [symbol] ₖh(k). (0.10)

The velocity spectrum is then found by tracking along straight lines in yₚ instead of xₚ as we did in (0.5):

$$\begin{matrix}\hat{p} ( v ) = {\vert {\sum }_{k}^{ } w ( k ) {y}_{p} ( {t}_{0} + k δ , {k}_{0} + k ) \vert}^{2} & ( 0.11 ) \\ = {\sum }_{{k}_{1} , {k}_{2}}^{ } {w}^{*} ( {k}_{1} ) w ( {k}_{2} ) {{y}_{p}}^{*} ( {t}_{0} + {k}_{1} δ , {k}_{0} + {k}_{1} ) & ( 0.12 ) \\ \\ {y}_{p} ( {t}_{0} + {k}_{2} δ , {k}_{0} + {k}_{2} ) , & \end{matrix}$$

and its expectation value is

$$\begin{matrix}〈 \hat{p} ( v ) 〉 = {\sum }_{{k}_{1} , {k}_{2}}^{ } {w}^{*} ( {k}_{1} ) w ( {k}_{2} ) 〈 {{y}_{p}}^{*} ( {t}_{0} + {k}_{1} δ , {k}_{0} + & ( 0.13 ) \\ {k}_{1} ) {y}_{p} ( {t}_{0} + {k}_{2} δ , {k}_{0} + {k}_{2} ) 〉 & \\ = {\sum }_{k}^{ } {w}_{2} ( k ) {R}_{{y}_{p}} ( kδ , k ) , & ( 0.14 ) \\ where k = {k}_{2} - {k}_{1} and & \\ {R}_{{y}_{p}} ( τ , m ) = {R}_{{x}_{p}} ( τ , m ) {⊗}_{m} {h}_{2} ( m ) & ( 0.15 ) \\ = {\sum }_{n} {R}_{{x}_{p}} ( τ , m - n ) {h}_{2} ( n ) . & ( 0.16 )\end{matrix}$$

This allows us to rewrite the velocity spectrum as

$$\begin{matrix}〈 \hat{p} ( v ) 〉 = {\sum }_{ k}^{ } {w}_{2} ( k ) {R}_{{x}_{p}} ( k δ , k ) {⊗}_{k} {h}_{2} ( k ) \\ = {\sum }_{n , m}^{ } {w}_{2} ( m ) {R}_{{x}_{p}} ( m δ , m - n ) {h}_{2} ( n ) . ( 0.18 )\end{matrix} \qquad ( 0.17 )$$

When measuring jet flow the sonographer typically tries to align the ultrasound beam with the jet direction to minimize the transversal component of the velocity, that is, vₜ=0. In this case b in (0.6) is constant, so the autocorrelation function becomes

Rₓ_{ₚ}(τ,m;v₀)=s₂(τ−mδᵥ_{₀}), (0.19)

Rₓ_{ₚ}(mδ,m−n;v₀)=s₂(mδ−(m−n)δᵥ_{₀}). (0.20)

Given a uniform velocity field we can insert (0.20) into (0.18) to obtain

$$\begin{matrix}〈 \hat{p} ( v \vert {v}_{0} ) 〉 = {\sum }_{m , n} {w}_{2} ( m ) \cdot {s}_{2} ( - m \frac{2 vT}{c} + ( m - n ) \frac{2 {v}_{0} T}{c} ) \cdot {h}_{2} ( n ) \\ = {{\sum }_{m}}^{ } {w}_{2} ( m ) {\sum }_{n}^{ } {s}_{2} ( m \frac{2 T}{c} ( {v}_{0} - v ) - n \frac{2 {v}_{0} T}{c} ) \cdot {h}_{2} ( n ) ( 0.22 )\end{matrix} \qquad ( 0.21 )$$

All the parameters of (0.22) are known system parameters, meaning that the expected power spectrum after clutter filtering [symbol] p(v) [symbol] can be modelled for any given jet velocity v₀. FIG. 13 shows an example of such a model for these settings:
Transmit frequency: 2.0 MHz
Pulse bandwidth: 0.5 MHz
Pulse repetition frequency: 15.9 kHz
Nyquist velocity vₙ: 3.06 m/s
Particle velocity: 2vₙ

This model can then be compared to the model without a clutter filter to determine the loss of signal power to the filter. This is plotted in FIG. 14 for a range of particle velocities and the settings above. The estimated signal power from the measurement can then be compensated using the modelled power loss to obtain an estimate of the true Doppler power. In this example, if the particle velocity is found to be 6 m/s the estimated Doppler power must be increased by 16.75 dB. This true Doppler power is then used in the quantitative Doppler calculations.

Combining this quantitative velocity information with the quantitative spatial information obtained by acquiring pulse-echo signals from a plurality of spatially adjacent regions and obtaining velocity spectra from all of them, a quantitative volume flow measurement can be obtained. Thus the present invention allows fluid flows to be quantitatively measured and analysed.

In particular embodiments, the present invention is applied in medical ultrasound for examining blood flow in the hearts and blood vessels (e.g. arteries and veins). The invention can provide both semi-quantitative and fully quantitative outputs for monitoring fluid flow in the heart valves, e.g. for assessing valvular regurgitations, or for assessing blood flow in stenotic arteries. Such quantitative measurements are extremely useful in assessing the severity of such pathologies and provide a useful tool for making diagnoses and for determining the extent to which further curative action should be taken.

FIG. 15 shows a further technique for producing a velocity spectrum. The technique of FIG. 15 is similar to the Velocity Matched Spectrum technique in that the sampling time is varied so as to match the radial velocity of the scatterer. However, FIG. 15 illustrates the use of a plurality of pulse-echo signals from spatially adjacent regions and where the velocity tracking also tracks the lateral (transverse) velocity of the scatterer.

FIG. 15 shows four spatially adjacent pulse-echo signals (1501, 1502, 1503, 1504). Each pulse-echo signal shows four large amplitude transmit pulses in time (1511, 1512, 1513, 1514), each followed by a low amplitude echo. The echoes are shown at delays dt1, dt2, dt3 and dt4 from pulse transmission. It can be seen that the echoes are getting closer in time to the transmit pulses, indicating that the scatterer is moving towards the transmitter (i.e. the round-trip pulse-echo time is reducing).

In an ordinary Velocity Matched Spectrum analysis, samples would be taken from a single pulse-echo signal (e.g. any one of 1501, 1502, 1503 and 1504) and when samples are taken at the times indicated by dt1, dt2, dt3 and dt4, the samples match the scatterer velocity and produce a peak in the output spectrum.

In FIG. 15, it can also be seen that the strongest echoes move laterally from one pulse-echo signal to another, indicating that the scatterer is moving laterally as well as radially. Therefore in the technique illustrated in FIG. 15, the Velocity Matched Spectrum samples are taken from different pulse-echo signals, thereby matching the lateral velocity of the scatterer and producing a higher peak in the output.

The specific example shown in FIG. 15 shows the strongest echo from pulse 1511 in Pulse-echo signal 1504, the strongest echo from pulse 1512 in Pulse-echo signal 1503, the strongest echo from pulse 1513 in Pulse-echo signal 1502 and the strongest echo from pulse 1514 in Pulse-echo signal 1501. The diagonal dashed line 1520 in FIG. 15 shows the radial and lateral Velocity Matched sampling which maximises the power output.

The technique illustrated in FIG. 15 may be used in a number of different circumstances to obtain a two or three dimensional velocity matched spectrum. It will be appreciated that this technique is particularly useful with the above-described techniques for semi-quantitative and quantitative analysis of pulse-echo signals from a plurality of spatially adjacent regions.

1-47. (canceled)

48. A method for monitoring fluid flow, the method comprising:
acquiring a pulse-echo signal from each of a plurality of spatially adjacent regions;
for each of the pulse echo signals, forming a velocity spectrum;
combining the plurality of velocity spectra into a composite data set which contains both spatial data and velocity data; and
displaying the composite data set so as to present at least one aspect of the velocity data with at least one aspect of the spatial data.

49. A method as claimed in claim 48, wherein the step of combining the plurality of velocity spectra into a composite data set comprises combining the spectra into a composite velocity spectrum.

50. A method as claimed in claim 49, wherein the composite velocity spectrum comprises a plurality of velocity bins each corresponding to a range of velocities, and, wherein the step of displaying the composite velocity spectrum includes displaying a representation of each velocity bin in the composite velocity spectrum with a colour that depends on the number of pulse-echo signals intersecting with the fluid flow.

51. A method as claimed in claim 49, wherein the composite velocity spectrum comprises a plurality of velocity bins each corresponding to a range of velocities and, in association with each velocity bin, the spectrum contains a representation of the number of individual region spectra in which the received signal power in that velocity bin is above a threshold value.

52. A method as claimed in claim 51, wherein the threshold value is different for different velocity bins.

53. A method as claimed in claim 50, further comprising:
displaying the composite velocity spectrum by plotting the velocity bins on a display such that a display characteristic of each velocity bin is determined from the number of individual region spectra in which the received signal power in that velocity bin is above a threshold value.

54. A method as claimed in claim 53, wherein the display characteristic is the colour of the plotted velocity bin.

55. A method as claimed in claim 49, wherein the composite velocity spectrum comprises a plurality of velocity bins each corresponding to a range of velocities,
wherein the plurality of regions are grouped into at least two groups, and
in association with each velocity bin, the composite velocity spectrum comprises a representation for each group of the total power in that velocity bin from all regions within that group.

56. A method as claimed in claim 55, wherein the representation for each group is weighted according to the inverse of the number of regions within that group.

57. A method as claimed in claim 55, further comprising:
displaying the composite velocity spectrum by plotting the velocity bins on a display such that a display characteristic of each velocity bin is determined from the total group powers of each group.

58. A method as claimed in claim 57, wherein the display characteristic is the colour of the plotted velocity bin.

59. A method as claimed in claim 58, wherein each group of regions is associated with a colour and wherein the colour of the plotted velocity bin of the composite velocity spectrum is determined by combining the colours of the plurality of groups in amounts based on the group total power for that velocity bin.

60. A method as claimed in claim 59, wherein there are two groups and the colours associated with the two groups are complementary colours.

61. A method as claimed in claim 55, wherein the regions are grouped into one group of one region and one group of all the remaining regions.

62. A method as claimed in claim 48, wherein the step of displaying the composite data set comprises:
for each region, plotting on a display at a spatial location corresponding to the spatial location of that region a representation of that region, wherein a display characteristic of the plotted region is determined from the velocity data associated with that region.

63. A method as claimed in claim 62, wherein the display characteristic is determined from the associated velocity data.

64. A method as claimed in claim 63, wherein the display characteristic is determined from the average or maximum velocity in the associated velocity data.

65. A method as claimed in claim 62, wherein the display characteristic is determined from the signal power corresponding to each velocity bin.

66. A method as claimed in claim 62, wherein the display characteristic is the colour of the plotted region.

67. A method as claimed in claim 66, wherein different colours are associated with different portions of the velocity spectrum.

68. Apparatus for monitoring fluid flow, comprising:
a transducer arranged to acquire a pulse-echo signal from each of a plurality of spatially adjacent regions; and
a processor arranged to
form a velocity spectrum for each of the pulse echo signals;
combine the plurality of velocity spectra into a composite data set which contains both spatial data and velocity data; and
display the composite data set so as to present at least one aspect of the velocity data with at least one aspect of the spatial data.

69. A software product comprising instructions which when executed by a computer cause the computer to:
acquire a pulse-echo signal from each of a plurality of spatially adjacent regions;
for each of the pulse echo signals, form a velocity spectrum;
combine the plurality of velocity spectra into a composite data set which contains both spatial data and velocity data; and
display the composite data set so as to present at least one aspect of the velocity data with at least one aspect of the spatial data.
